(19) United States
(12) Patent Application Publication
(10) Pub. No.: US 2012/0029355 A1
(43) Pub. Date: Feb. 2, 2012

(54) Bone Sonometer

(75) Inventors: Tal Marom, Rishon LeZion (IL); Gilad Zamir, Kiryat Ono (IL)

(73) Assignee: BEAMMED LTD., Petah Tikva (IL)

(21) Appl. No.: 13/263,160

(22) PCT Filed: Apr. 7, 2010
(86) PCT No.: PCT/IL10/00292

§ 371 Date: Oct. 6, 2011

Related U.S. Application Data
(60) Provisional application No. 61/167,176, filed on Apr. 7, 2009.

(51) Int. Cl. A61B 8/00

(52) U.S. Cl. 600/442; 600/459

(57) ABSTRACT

A probe device is for performing a bone density measurement is disclosed. The device comprises at least one ultrasound source for providing ultrasonic pulses; a plurality of ultrasound detectors for measuring the differences in arrival times of said ultrasonic pulses; at least one dedicated data processing element adapted for determining differences in arrival times of ultrasonic pulses; means for transferring data from said at least one measurement transducer to said at least one dedicated data processing element; and communication means adapted to transmit data from said dedicated data processing element to a non-dedicated computing means. Unlike systems known in the art, the measurements of the times of arrival of the ultrasonic pulses are performed within the probe itself, obviating the need for a dedicated computer system or measurement card.

### FIELD OF THE INVENTION

The present invention relates to systems for bone sonometery e.g., densitometry and methods for the use thereof.

### BACKGROUND OF THE INVENTION

Bone mass loss is a widespread medical condition, appearing with particular frequency in the elderly and in women. The gradual depletion of a person's bone mass can make the bone prone to fracture and/or deformation and cause numerous accompanying adverse effects, including pain and discomfort. The condition of osteoporosis manifests itself as a decrease in bone tissue mass and often leads to fractures of the vertebrae, hip, femur, and distal end of the wrist bone.

The World Health Organization defines four diagnostic categories: normal, osteopenia, osteoporosis, and established osteoporosis, and further defines those categories using diagnostic value ranges. Currently, within the United States, osteoporosis affects about 20-25 million people. By age 80, the percentage of women with normal bone density decreases to 15%. Due to this condition, one out of every six women will have a hip fracture and one out of every three women will have a vertebral fracture during their lifetime.

Certain medical evaluations can be conducted to determine whether osteoporosis may be present in a patient, including the examination of a patient's height and weight, investigating the presence of pain or deformity in the bones, and identifying underlying medical illnesses using blood cell counts, PTH blood tests, mineral content (calcium, phosphorus, among others), a thyroid test, and vitamin D levels. Once major deterioration has occurred, it is difficult to restore the lost bone. Thus, therapeutic efforts must be directed towards early recognition of the progressive disease so that treatment can be instituted before irreversible structural damage occurs.

One approach to diagnosing the existence of osteoporosis in a patient or a patient's susceptibility to bone-loss related ailments, such as bone fractures or osteopenia, is to test a patient's bone and compare the values to established references. Various devices may be used. Ultrasound techniques are advantageous in that they are non-invasive and operate on the principle that the velocity and attenuation of the signal through the patient's bone is a measure of the characteristics of the bone.

Certain protocols do exist for the diagnosis and treatment of osteoporosis. For example, it is recommended that 1) persons over the age of 65 should have a bone mineral density (BMD) test; 2) persons over the age of 50 with at least one major, or two minor, risk factors should have a BMD test; 3) postmenopausal women with risk factors for fracture should have a BMD test; 4) higher intakes of calcium and vitamin D are recommended, particularly in adults over 50 (calcium 1500 mg/day and vitamin D 800 IU/day); and 5) people should participate in exercise, particularly weight-bearing exercises such as brisk walking, running or dancing. Formal protocols, such as the Osteoporosis Risk Assessment Instrument (ORAI) and Simple Calculated Osteoporosis Risk Estimation (SCORE), provide more defined algorithms for identifying persons at risk for osteoporosis based on variables such as the person's age, weight, and estrogen use. However the precision of these tests is not specified, nor is the frequency with which such tests be carried out.

Obviously to properly initiate, conduct, and monitor the effects of a treatment and/or prevention regimen, sufficient knowledge of the state of a person's bone mass, along with rate of increase or decrease is preferred. Current treatment and/or prevention protocols fail to adequately account for or incorporate such information.

Although exercising, dietary, and other methods of prevention may exist, there is a need to integrate these various preventive and/or treatment measures with bone measurement techniques to create an integrated osteoporosis treatment protocol. There is also a need for improved methods and systems to determine changes in bone mass in a short period of time, to examine patients and analyze bone deformities to comprehensively assess bone material, and to provide a practitioner with bone data to predict future bone characteristics, to prevent bone loss, to avoid fractures, and to increase bone density.

In the prior art a variety of systems for bone densitometry have been disclosed. These are often based on various x-ray based techniques including single and dual-photon absorptiometry (SPA and DPA), quantitative computed tomography (QCT), and single- and dual-energy absorptiometry (SXA and DXA). Other methods of measurement include and spinal and peripheral quantitative x-ray computed tomography (QCT and PQCT). All are capable of evaluating bone mineral density (BMD) as the test parameter. The result is given as an absolute scale, and also relative to population reference values. All of these methods expose the patient and operator to x-ray radiation. Due to the ionizing nature of X-radiation the frequency with which such measurements may be carried out is limited, to limit dosage of ionizing radiation. For this reason alternative methods such as ultrasonic methods bear certain advantages over X-ray methods.

As a further example, x-ray based equipment requires a licensed technician to operate the equipment due to the ionizing radiation hazards involved. In addition, this equipment is structurally large, requiring an x-ray source, detector, power supply, cooling equipment, as well as an examination area usually consisting of a bed.

Biochemical bone markers can be used to estimate the rate of bone dissolution and/or bone formation; as such they are considered an indirect measurement for bone assessment. Nevertheless, they can be used for estimating the rate of change of bone mass, useful for example in evaluating response to treatment.

The most common method for bone mineral density (BMD) measurement is called DXA or dual-energy x-ray absorptiometry. Most commonly, DXA test results are compared to the ideal or peak bone mineral density of a healthy 30-year-old adult. The difference between a subjects bone density and this ideal, as measured in standard deviations, is called a t-score. A score of 0 means one's BMD is equal to the norm for a healthy young adult. The more standard deviations below 0, indicated as negative numbers, the lower one's BMD and the higher the risk of fracture.

A t-score between −1 and −2.5 indicates low bone mass, although not low enough to be diagnosed with osteoporosis. A t-score of −2.5 or lower indicates osteoporosis. The greater the negative number, the more severe the osteoporosis.

The BMD of a subject may also be compared to that of a typical individual whose age is matched to the subject's. This comparison is called the z-score. Because low BMD is common among older adults, comparisons with the BMD of a typical individual whose age is matched to yours can be misleading. Therefore, the diagnosis of osteoporosis or low bone mass is typically based on the subject's t-score. However, a z-score can be useful for determining whether an underlying disease or condition is causing bone loss.

Amongst known problems with the DXA method are its relatively low accuracy, the radiation dosage involved in the measurement, and the size of the apparatus.

It is of paramount importance to be able to detect small changes in bone density, since bone mass once lost is nearly impossible to replace. The expected rate of bone loss can be rather low: for example, bone was lost from lumbar spine at a rate of 0.59% per year in a study of 297 women aged 50-81 (“Rate of bone loss from lumbar spine in women with distal forearm fracture”, Peel et al, BMJ 312:1457, 8 Jun. 1996). In a study of 178 postmenopausal women, the rate of bone loss over two years was compared to that over twelve years. This comparison is shown in FIG. 10, where the actual bone loss over 12 years is shown on the y-axis, vs. the actual bone loss over 22 years is shown on the x-axis. Actual rates range from a high of about 3% per year, and therefore any method designed to measure bone loss over periods of a year or less must have an error significantly less than the maximum 3% loss to be measured. It is known that DXA and SPA cannot provide such accurate measurements; for example the coefficient of variation for repeat measurements by DEXA was found to be 1.2% and that for SPA 1.6% (Weinstein et al, “Dual-energy X-ray absoptiometry versus single photon absorptiometry of the radius”, Calcified Tissue International V 49 N 5 Sep. 1991). The error in accuracy of single photon absoptiometry has been estimated at 6% and the error in precision at 2-4% [Blake et al, “The evaluation of osteoporosis: dual energy X-ray absorptiometry and ultrasound in clinical practice.” London: Martin Dunitz, 1999] Even with an error for DXA of 1.2%, this is nearly half the greatest change expected in one year, and it is clear that more reliable results would be useful. This is especially so given that repeated measurements (which in principle could be used to find a reliable average value despite the scatter of individual measurements) cannot be used in the case of X-ray based measurement.

An alternative approach to BMD determination has been the use of quantitative ultrasound (QUS) methods. QUS is an accepted method for the assessment of bone status, primarily because it offers quick, relatively low cost results without the radiation associated with other traditional techniques such as radiography, x-ray absorptiometry and computed tomography.

Sound energy consists of alternating cycles of compression and rarefaction of the medium through which it is transmitted. Audible sound for humans is in the range of approximately 20 Hz to 20,000 Hz (20 kHz). Ultrasound refers to a range of frequencies that begins at the high-frequency end of the audible range and extends into the Megahertz range. The propagation of ultrasound through a medium, its speed, its dispersion and the attenuation of signal strength are strongly influenced by the physical properties of that medium. For example, the speed of propagation increases with the density of the medium and its modulus of elasticity (Young's Modulus). Moreover, the microstructure of the medium, as well as macro-structures on the order of a wavelength of the ultrasound, affects the speed.

The speed of sound depends, among other factors, on the density of the medium through which it is traveling. At a typical ultrasonic frequency of 1.25 MHz, the acoustic signal travels much faster through the relatively dense, cortical layer of the bone than through the trabecular layer, e.g., approximately 4000 m s⁻¹ vs. 1800 m s⁻¹. The signal travels through soft tissue much more slowly than through either type of bone, at a speed of about 1540 m s⁻¹.

Parameters that can be determined using ultrasound include the speed of sound and the attenuation of the ultrasound signal as it penetrates bone and tissue. These parameters provide general characteristics relating to bone density, bone strength, and the risk of future fracture.

Ultrasonic methods generally employ a pair of opposed spaced ultrasonic transducers held within a clamping apparatus closely adjacent to the bone being analyzed. These ultrasonic transducers include piezoelectric elements shaped to direct signals through the bone encompassed, for example, in the heel and finger of the subject being tested. A pulse generator is coupled to one of the transducers and generates an electric pulse for causing the transducers to generate an ultrasonic sound wave that is directed through the bone structure to the other transducer. The time that it takes the ultrasonic wave to travel through the bone structure being examined is measured, and this duration measurement is transformed into a speed measurement, which correlates with bone density.

Improved methods for bone densitometry by use of ultrasound velocity measurements were disclosed for example in U.S. Pat. No. 6,234,969 (henceforth '969) and U.S. Pat. No. 6,135,964. In these devices, differences in sound conduction velocity are utilized to determine bone density. In addition to being entirely benign and therefore usable at any arbitrary ultrasonic frequency, the use of sound velocity also provides an indication of bone porosity that correlates with bone strength, which X-ray measurements do not. The '969 patent provides a foot well, sending and receiving ultrasound transducers, and a controller. The use of a foot well limits the portability of the device and the possible applications thereof, however.

Portable devices using ultrasound technology have generally been provided in the form of portable units including a computer, bone densitometer, and signal processing equipment located on a drop-in card in the computer. Since the device requires proprietary processing equipment on this drop-in card, the portability of the unit is limited by the requirement to provide a computer containing the proper card along with the measurement head.

Furthermore, current methods are based on single point methods that indicate only a gross overall measure of bone density and strength, and cannot discover small points of compromised bone density.

A need thus exists for a compact and inexpensive system that may be installed in a practitioner's office or like location and that reduces the time necessary to determine bone resorption or formation and to permit a practitioner to diagnose and manage bone related disorders.

Hence, an improved portable system and method of bone densitometry, protocols for such densitometry, and methods for carrying out densitometry remain a long felt need.

### SUMMARY OF THE INVENTION

The present invention fulfills this long-felt need. It comprises a system and method for bone density measurement based on sound velocity differences in media of different density in which the means for measuring differences in arrival times of ultrasonic pulses are located within a dedicated detector located either within the measuring head or within a special probe. While the bone sonometry measurements are being made, the measuring head or probe is located near the body of the patient. Thus, the invention obviates the need for a special drop-in card, and enables performance of bone sonometry measurements without any need for a dedicated computer system.

It is thus an object of the present invention to disclose a probe device for performing a bone density measurement, said device comprising (a) at least one ultrasound source for providing ultrasonic pulses; (b) a plurality of ultrasound detectors for measuring the differences in arrival times of said ultrasonic pulses; (c) at least one dedicated data processing element adapted for determining differences in arrival times of ultrasonic pulses; (d) means for transferring data from said at least one measurement transducer to said at least one dedicated data processing element; and (e) communication means adapted to transmit data from said dedicated data processing element to a non-dedicated computing means. It is within the essence of the invention wherein analysis of said differences in said arrival times is performed by said non-dedicated computing means.

It is a further object of this invention to disclose such a device, wherein said probe comprises an array of said ultrasound sources and detectors adapted for being placed on or around a portion of a subject's body.

It is a further object of this invention to disclose such a device, wherein said probe is flexible.

It is a further object of this invention to disclose a self-contained ultrasound bone sonometer device for performing multi-site bone density measurements of a region of a subject's body, said device comprising: (a) a measurement head, said measurement head comprising: (1) an ultrasound source comprising an array of ultrasound transducers adapted for providing ultrasonic pulses; (2) at least one measurement transducer adapted to measure the difference in arrival times of ultrasonic pulses; and (3) at least one dedicated data processing element adapted for determining differences in arrival times of ultrasonic pulses; (b) means for transferring data from said at least one measurement transducer to said at least one dedicated data processing element; and (c) communication means adapted to transmit data from said dedicated data processing element to a non-dedicated computing means. It is within the essence of the invention wherein a multi-site bone density measurement is obtained from said determination of differences in arrival times of ultrasonic pulses, and further wherein said determination is performed within said measurement head.

It is a further object of this invention to disclose an improved ultrasound bone sonometer device for multi-site bone density measurements, said device comprising: (a) a cuff-like measurement head adapted to enclose at least part of the circumference of a region of a subject's body, said measurement head comprising an ultrasound source comprising an array of ultrasound transducers adapted for providing ultrasonic pulses; (b) a dedicated data processing element; and (c) communication means adapted to transmit data from said dedicated processing element to a non-dedicated computing means; wherein the improvement consists of the placement of (a) at least one measurement transducer adapted to measure the difference in arrival times of ultrasonic pulses and (b) at least one dedicated data processing element adapted for determining differences in arrival times of ultrasonic pulses within said measurement head.

It is a further object of this invention to disclose a device as defined in any of the above, wherein said dedicated data processing element is an ASIC.

It is a further object of this invention to disclose a device as defined in any of the above, wherein said non-dedicated computing means is selected from the group consisting of server, PC, mobile communication means, workstation, personal digital assistant, laptop computer, desktop computer, tablet computing device, and any combination thereof.

It is a further object of this invention to disclose a device as defined in any of the above, wherein said ultrasound source is adapted to provide (i) a first ultrasonic pulse transmitted along a transmission path extending from a first location to a second location, said transmission path comprising passage from said first location through soft tissue generally toward at least one bone, reflection from said at least one bone, and passage through said soft tissue generally away from said at least one bone and generally towards said second location; and, (ii) a second ultrasonic pulse transmitted along a transmission path from said first location to a third location, said transmission path comprising passage from said first location through soft tissue generally toward at least one bone, passage over the surface of said at least one bone, and passage through said soft tissue generally away from said at least one bone and generally towards a third location.

It is a further object of this invention to disclose a device as defined in any of the above, wherein said non-dedicated computing means comprise software adapted to perform at least one task chosen from the group consisting of (a) calculating the acoustic velocity of said ultrasonic pulses in said bone from the distances between said first, second, and third locations, and the difference between the time of propagation of an ultrasonic pulse from said first location to said second location and the time of propagation of an ultrasonic pulse from said first location to said third location; (b) displaying the results of said acoustic velocity measurements; and (c) storing the results of said acoustic velocity measurements.

It is a further object of this invention to disclose a device as defined in any of the above, wherein said bone density measurement is presented in terms selected from the group consisting of BUA (Broadband Ultrasound Attenuation); SOS (Speed Of Sound); EPO (Expected Age of Osteoporosis); PAB (Physiological Age of Bone); TTO (Time To Osteoporosis); STI (Strength Index); RRF (Relative Risk of Fracture); pediatric capabilities; and (RFI) Risk Fracture Index.

It is a further object of this invention to disclose a device as defined in any of the above, wherein said communication means are adapted to transmit data according to a communication protocol selected from the group consisting of: ZigBee, Bluetooth, IRDA, 3G, 4G, HSDPA, 3.9G, IEEE 802.11, IEEE 802.15.x, IEEE 802.16, HiperLAN, WiMAX, and GSM.

It is a further object of this invention to disclose a device as defined in any of the above, wherein said communication means comprise a data connection selected from the group comprising USB, RS232, parallel cable, and wireless.

It is a further object of this invention to disclose a device as defined in any of the above, wherein said communication means comprise networking means selected from the group comprising LAN, wireless LAN, PAN, and wireless PAN.

It is a further object of this invention to disclose a device as defined in any of the above, wherein said communication means comprise communication methods selected from the group comprising CDMA, packet radio, and GPRS.

It is a further object of this invention to disclose a device as defined in any of the above, wherein said communication means comprise means for transmitting data in at least one frequency range chosen from the group comprising visible light, infrared, microwave, and radiofrequency.

It is a further object of this invention to disclose a device as defined in any of the above, further comprising a communicable database adapted for storage and retrieval of bone density readings.

It is a further object of this invention to disclose a device as defined in any of the above, wherein said device is adapted to image or otherwise provide an analysis of at least one site within said measured region of a subject's body chosen from the group consisting of rigid sites and semi-rigid sites.

It is a further object of this invention to disclose such a device, wherein said rigid site is a bone, bone-like biological tissue or region of a subject's body thereof.

It is a further object of this invention to disclose such a device, wherein said semi-rigid site is a cartilage, cartilage-like biological tissue or region of a subject's body.

It is a further object of this invention to disclose a device as defined in any of the above, wherein said non-dedicated computing means are provided with processing software for analyzing said differences in said arrival times.

It is a further object of this invention to disclose a device as defined in any of the above, wherein said array of ultrasound sources and said ultrasound detector are located substantially on the same side of said region.

It is a further object of this invention to disclose a device as defined in any of the above, wherein said array of ultrasound sources and said ultrasound detector are located on opposite sides of said region.

It is a further object of this invention to disclose a device as defined in any of the above, wherein said array of ultrasound sources are constructed according to a phased array model.

It is a further object of this invention to disclose a device as defined in any of the above, wherein said ultrasound detectors are adapted for measuring the differences in arrival times of said ultrasonic pulses.

It is a further object of this invention to disclose a device as defined in any of the above, wherein said subject is selected from a group consisting of postmenopausal women, patients having “fragility” fracture, patients given corticosteroid medications, patients with thyroid disease, and patients of the age range of about 50 to about 65 years old.

It is a further object of this invention to disclose a sonometer device for early detection of bone density changes in a subject in the age ranging from about 50 to about 65; said device comprising means for providing periodic bone density (PBD) readings with low precision error (LPE); wherein said precision error of said periodic bone density readings is of at least about an order of magnitude less than the expected annual change (EAC) in said subject, such that said early detection of said bone changes is provided.

It is a further object of this invention to disclose such a device, wherein said precision error of said periodic bone density readings is less than 1% of the EAC in said subject, such that said early detection of said bone changes is provided.

It is a further object of this invention to disclose such a device, wherein said means for providing PBD readings comprises: (a) an array of ultrasound sources adapted for providing ultrasonic pulses; (b) at least one measurement transducer adapted to measure the difference in arrival times of ultrasonic pulses; and (c) at least one dedicated data processing element adapted for determining differences in arrival times of ultrasonic pulses.

It is a further object of this invention to disclose such a device, wherein said means for providing periodic bone density readings additionally comprises: (a) at least one ultrasound source adapted for providing ultrasonic pulses; (b) a plurality of ultrasound detectors adapted for measuring the differences in arrival times of said ultrasonic pulses; and (c) processing means for analyzing said differences in said arrival times.

It is a further object of this invention to disclose a device as defined in any of the above, wherein said sonometer is a bone densitometer.

It is a further object of this invention to disclose a device as defined in any of the above, additionally comprising means for limiting the amplitudes of said ultrasonic pulses to less than about 520 mV.

It is a further object of this invention to disclose a device as defined in any of the above, additionally comprising means for limiting the amplitudes of said ultrasonic pulses to about 400 mV.

It is a further object of this invention to disclose a device as defined in any of the above, additionally comprising means for normalizing the amplitudes of the signals received by said measurement head to about 400 mV.

It is a further object of this invention to disclose a method for performing bone density measurements, comprising steps of: (a) obtaining a portable ultrasound bone sonometer, comprising a measurement head provided with at least one ultrasound source, a plurality of ultrasound detectors, and at least one dedicated data processing element adapted for determining differences in arrival times of ultrasonic pulses; (b) transmitting a plurality of ultrasonic pulses by said ultrasound source; (c) detecting said a plurality of ultrasonic pulses by said detectors; (d) measuring the differences in arrival times of said ultrasonic pulses; (e) communicating said measurements of arrival times to at least one non-dedicated computing means; and (f) analyzing said differences in said arrival times. It is within the essence of the invention wherein said step of analyzing is performed by said non-dedicated computing means.

It is a further object of this invention to disclose a method for performing multi-site bone density measurements of a region of subject's body, said method comprising steps of: (a) obtaining an ultrasound bone sonometer, said sonometer comprising (1) a cuff-like measurement head adapted to enclose at least part of the circumference of said region of said subject's body, said measurement head comprising: (i) an array of ultrasound sources adapted for providing ultrasonic pulses; (ii) at least one measurement transducer adapted to measure the difference in arrival times of ultrasonic pulses; and (iii) at least one dedicated data processing element adapted for determining differences in arrival times of ultrasonic pulses; (2) means for transferring data from said at least one measurement transducer to said at least one dedicated data processing element; and (3) means for transmitting data from said dedicated data processing element to a non-dedicated computing means; (b) encircling said region of said subject's body by said flexible measurement head; (c) transmitting ultrasonic pulses from said ultrasound sources to said multiple sites encircled within said region; (d) detecting said ultrasonic pulses from said multiples sites encircled within said region; and (e) measuring the acoustic velocity of said ultrasonic pulses, thereby simultaneously performing said multi-site bone density measurements. It is within the essence of the invention wherein said step of performing said multi-site bone density measurements is provided by said step of encircling said region of said subject's body by said flexible measurement head without having to relocate said measurement head from one site to another.

It is a further object of this invention to disclose such a method as defined in any of the above, wherein said step of transmitting a plurality of ultrasonic pulses by said ultrasound source additionally comprises transmitting (i) a first ultrasonic pulse transmitted along a transmission path extending from a first location to a second location, said transmission path comprising passage from said first location through soft tissue generally toward at least one bone, reflection from said at least one bone, and passage through said soft tissue generally away from said at least one bone and generally towards said second location; and, (ii) a second ultrasonic pulse transmitted along a transmission path from said first location to a third location, said transmission path comprising passage from said first location through soft tissue generally toward at least one bone, passage over the surface of said at least one bone, and passage through said soft tissue generally away from said at least one bone and generally towards a third location.

It is a further object of this invention to disclose such a method, wherein said step of measuring the acoustic velocity of said ultrasonic pulses additionally comprises a step of calculating the acoustic velocity of said ultrasonic pulses in said bone from the distances between said first, second, and third locations and the difference in time of propagation of an ultrasonic pulse from said first location to said second location and the time of propagation of an ultrasonic pulse from said first location to said third location.

It is a further object of this invention to disclose such a method, additionally comprising a step of displaying and storing said acoustic velocity measurements on said non-dedicated computing means.

It is a further object of this invention to disclose such a method, additionally comprising a step of selecting said computing means from the group consisting of: personal digital assistant, laptop computer, desktop computer, and tablet computing device.

It is a further object of this invention to disclose such a method as defined in any of the above, additionally comprising a step of presenting said bone density measurements in terms of a z-score.

It is a further object of this invention to disclose such a method as defined in any of the above, additionally comprising a step of presenting said bone density measurements in terms of a t-score.

It is a further object of this invention to disclose such a method as defined in any of the above, additionally comprising a step of presenting said bone density measurements in terms selected from a group consisting of BUA (Broadband Ultrasound Attenuation); SOS (Speed Of Sound); EPO (Expected Age of Osteoporosis); PAB (Physiological Age of Bone); TTO (Time To Osteoporosis); STI (Strength Index); RRF (Relative Risk of Fracture); pediatric capabilities; and (RFI) Risk Fracture Index.

It is a further object of this invention to disclose such a method as defined in any of the above, wherein said step of transmitting a plurality of ultrasonic pulses by said ultrasound source additionally comprises (i) transmitting a first ultrasonic pulse transmitted along a transmission path extending from a first location to a second location, said transmission path comprising passage from said first location through soft tissue generally toward at least one bone, reflection from said at least one bone, and passage through said soft tissue generally away from said at least one bone and generally towards said second location; and, (ii) transmitting a second ultrasonic pulse transmitted along a transmission path from said first location to a third location, said transmission path comprising passage from said first location through soft tissue generally toward at least one bone, passage over the surface of said at least one bone, and passage through said soft tissue generally away from said at least one bone and generally towards a third location.

It is a further object of this invention to disclose such a method as defined in any of the above, wherein said step of analyzing additionally comprises a step of calculating the acoustic velocity of said ultrasonic pulses in said bone from the distances between said first, second, and third locations, and the difference between the time of propagation of an ultrasonic pulse from said first location to said second location and the time of propagation of an ultrasonic pulse from said first location to said third location.

It is a further object of this invention to disclose such a method as defined in any of the above, wherein said step of analyzing additionally comprises steps of (a) displaying the results of said acoustic velocity measurements; and (b) storing the results of said acoustic velocity measurements.

It is a further object of this invention to disclose such a method as defined in any of the above, wherein said step of communicating additionally comprises a step of selecting a communication means adapted to transmit data according to a communication protocol selected from the group consisting of: ZigBee, Bluetooth, IRDA, 3G, 4G, HSDPA, 3.9G, IEEE 802.11, IEEE 802.15.x, IEEE 802.16, HiperLAN, WiMAX, and GSM.

It is a further object of this invention to disclose such a method as defined in any of the above, wherein said step of communicating additionally comprises a step of selecting a communication means, said communication means comprising a data connection selected from the group comprising USB, RS232, parallel cable, and wireless.

It is a further object of this invention to disclose such a method as defined in any of the above, wherein said step of communicating additionally comprises a step of selecting a communication means, said communication means comprising networking means selected from the group comprising LAN, wireless LAN, PAN, and wireless PAN.

It is a further object of this invention to disclose such a method as defined in any of the above, wherein said step of communicating additionally comprises a step of selecting a communication means, said communication means comprising communication methods selected from the group comprising CDMA, packet radio, and GPRS.

It is a further object of this invention to disclose such a method as defined in any of the above, wherein said step of communicating additionally comprises steps of: (a) selecting a communication means adapted to transmit data in at least one frequency range chosen from the group comprising visible light, infrared, microwave, and radiofrequency; and (b) transmitting data in said frequency range.

It is a further object of this invention to disclose such a method as defined in any of the above, additionally comprising a step of calibrating said measurement.

It is a further object of this invention to disclose such a method as defined in any of the above, wherein said step of calibrating said measurement comprises a step of measuring the propagation time of an ultrasonic pulse through a known length of a medium in which the speed of propagation of said ultrasonic pulse is known.

It is a further object of this invention to disclose a method for early detection of bone density changes in subjects characterized in the age range of about 50 to about 65, comprising a step of performing periodic bone density readings at locations selected from the group consisting of the radius, the phalanx, the metatarsal and any combination thereof; wherein said readings are characterized by an LPE, and further wherein said precision error of said periodic bone density readings is at least about one order of magnitude less than the EAC, such that said early detection of said bone changes is provided.

It is a further object of this invention to disclose such a method for early detection of bone density changes, wherein said step of periodic bone density readings additionally comprises steps of: (a) transmitting a first ultrasonic pulse transmitted along a transmission path extending from a first location to a second location, said transmission path comprising passage from said first location through soft tissue generally toward at least one bone, reflection from said at least one bone, and passage through said soft tissue generally away from said at least one bone and generally towards said second location; (b) transmitting a second ultrasonic pulse transmitted along a transmission path from said first location to a third location, said transmission path comprising passage from said first location through soft tissue generally toward at least one bone, passage over the surface of said at least one bone, and passage through said soft tissue generally away from said at least one bone and generally towards a third location; and (c) calculating the acoustic velocity of ultrasound in said bone from on the distances between said first, second, and third locations and the difference between the time of propagation of an ultrasonic pulse from said first location to said second location and the time of propagation of an ultrasonic pulse from said first location to said third location.

It is a further object of this invention to disclose such a method for early detection of bone density changes, additionally comprising steps of: (a) limiting the amplitude of said ultrasonic pulses to less than 520 mV; and (b) normalizing the probes to approximately 400 mV.

It is a further object of this invention to disclose such a method for early detection of bone density changes, additionally comprising a step of selecting said subjects from a group consisting of postmenopausal women, patients having “fragility” fracture, patients given corticosteroid medications, patients with thyroid disease, patients of the age range of about 50 to about 65 years old.

It is a further object of this invention to disclose such a method for early detection of bone density changes, additionally comprising a step of performing said periodic bone density readings at intervals selected from the group consisting of quarterly, biannually, and annually.

It is a further object of this invention to disclose a method for measuring bone age comprising steps of: (a) providing a compact measurement head adapted to transmit an acoustic energy into the body of a subject; (b) receiving an acoustic signal from one or more structures including an ossification-actuated skeletal structure or a cranial structure that changes with age, responsive to said transmitted acoustic energy; (c) providing computing means adapted for analyzing the acoustic signal to determine at least one effect of said structure on said signal; (d) providing communications means adapted to transmit acoustic signal information from said measurement head to said computing means; and (e) estimating the age of the structure from said determined effect with said computing means. It is within the essence of the invention wherein bone age measurements are obtained in a system with separate measurement and computation means, allowing increased portability of said measurement head.

It is a further object of this invention to disclose a method for increasing the number of patients at high risk for bone fragility on whom bone density measurements are made, comprising steps of: (a) providing a bone sonometer adapted to perform bone fragility measurements; and (b) locating said bone sonometer at an accessible location, said accessible location selected from the group consisting of pharmacies, clinics, medical vehicles, ambulances, hospitals, medical departments, homes, points of care, pre-hospital sites, sports clubs, exercise facilities, stadiums, government offices, post offices, relief agencies, workout gyms, malls, and cosmetic counters; and further wherein said number of said bone fragility high risk patients on whom bone density measurements are made is increased by the increased accessibility of said bone sonometer.

It is a further object of this invention to disclose a method for increasing the frequency of bone density measurements performed on a patient, comprising steps of: (a) providing a bone sonometer adapted to perform bone fragility measurements; and (b) locating said bone sonometer at an accessible location, said accessible location selected from the group consisting of pharmacies, clinics, medical vehicles, ambulances, hospitals, medical departments, homes, points of care, pre-hospital sites, sports clubs, exercise facilities, stadiums, government offices, post offices, relief agencies, workout gyms, malls, and cosmetic counters; and further wherein said frequency of bone density measurements is increased by the increased accessibility of said bone sonometer.

It is a further object of this invention to disclose a method for increasing the number of patients on whom bone status measurements are made, comprising steps of: (a) providing a bone sonometer adapted for performing at least one measurement chosen from the group consisting of bone fragility measurements, bone density measurements, bone strength measurements, and bone age measurements; (b) providing said bone sonometer with multifunctional multipurpose screening means; (c) locating said bone sonometer at accessible locations; (d) analyzing at least one rigid or semi-rigid site within a measured region of the body of a patient with said bone sonometer; and (e) screening a preset matter with said screening means, wherein said accessible location is selected from the group consisting of pharmacies, clinics, medical vehicles, ambulances, hospitals, medical departments, homes, points of care, pre-hospital sites, sports clubs, exercise facilities, stadiums, government offices, post offices, relief agencies, workout gyms, malls, and cosmetic counters; and further wherein said number of patients at high risk for bone fragility on whom bone status measurements are made is increased by the accessibility of said bone sonometer.

It is a further object of this invention to disclose a method for increasing the frequency of bone status measurements performed on a patient, comprising steps of: (a) providing a bone sonometer adapted for performing at least one measurement chosen from the group consisting of bone fragility measurements, bone density measurements, bone strength measurements, and bone age measurements; (b) providing said bone sonometer with multifunctional multipurpose screening means; (c) locating said bone sonometer at accessible locations; (d) analyzing at least one rigid or semi-rigid site within a measured region of the body of a patient with said bone sonometer; and (e) screening a preset matter with said screening means; wherein said accessible location is selected from the group consisting of pharmacies, clinics, medical vehicles, ambulances, hospitals, medical departments, homes, points of care, pre-hospital sites, sports clubs, exercise facilities, stadiums, government offices, post offices, relief agencies, workout gyms, malls, and cosmetic counters; and further wherein said frequency of bone status measurements is increased by the accessibility of said bone sonometer.

It is a further object of this invention to disclose such a method as defined in any of the above, wherein said bone sonometer is a bone densitometer.

It is a further object of this invention to disclose such a method as defined in any of the above, wherein said patient is at high risk of bone fragility.

### BRIEF DESCRIPTION OF THE DRAWINGS

In order to understand the invention and to see how it may be implemented in practice, a plurality of embodiments will now be described, by way of non-limiting example only, with reference to the accompanying drawings, in which:

FIG. 1 presents a set of ultrasonic transceivers as described in prior art;

FIG. 2 presents a matrix of ultrasound transceivers as described in prior art;

FIG. 3 illustrates a mini-OMNI sonometer according to different embodiments of the present invention;

FIG. 4 presents an ultrasound measurement system according to one embodiment of the present invention;

FIG. 5 presents an ultrasound measurement kit according to one embodiment of the present invention;

FIG. 6 depicts the moving average of the SOS results as a function of age;

FIG. 7 presents the SOS distribution by study group within a particular clinical study;

FIG. 8 illustrates the SOS distribution by study group within a second clinical study;

FIG. 9 illustrates a bone age measurement method of prior art; and,

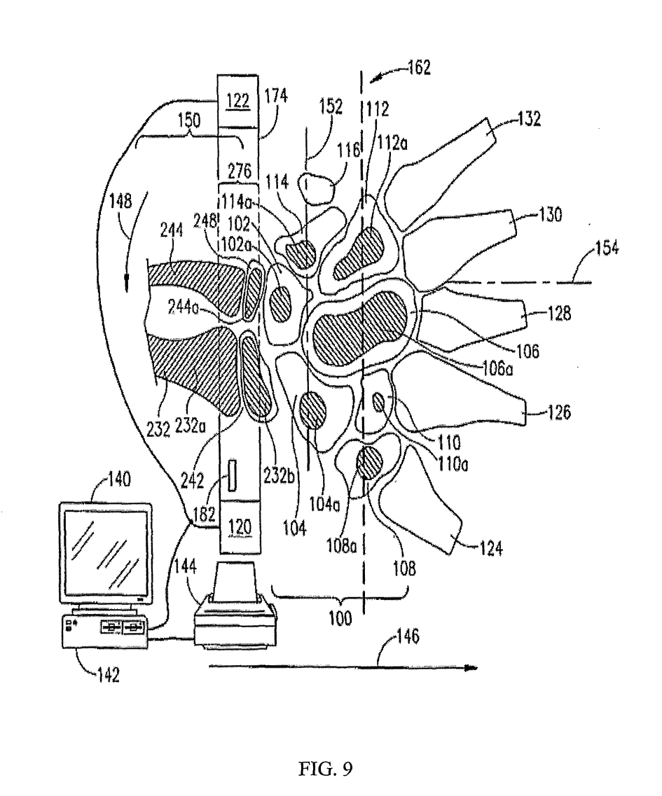

FIG. 10 presents a comparison between the rate of bone loss over two years and the rate of bone loss over twelve years in a study of 178 postmenopausal women.

### DETAILED DESCRIPTION OF THE PREFERRED EMBODIMENTS

The following description is provided in order to enable any person skilled in the art to make use of the invention and sets forth the best modes contemplated by the inventor of carrying out this invention. Various modifications, however, will remain apparent to those skilled in the art, since the generic principles of the present invention have been defined specifically to provide an improved bone densitometer and system for use thereof. In the following detailed description, numerous specific details are set forth in order to provide a thorough understanding of embodiments of the present invention. However, those skilled in the art will understand that such embodiments may be practiced without these specific details. Reference throughout this specification to “one embodiment” or “an embodiment” means that a particular feature, structure, or characteristic described in connection with the embodiment is included in at least one embodiment of the invention.

The distribution of bone density among the individuals in the general population is Gaussian (normal), and hence an individual's bone density relative to the average bone density can be reported meaningfully in terms of standard deviations. As used herein, the term “t-score” refers to the number of standard deviations that a given subject's bone density differs from the average for a young adult at peak bone density. The t-score has also been used to define osteoporosis. For example, based on the increasing risk of fracture with decreasing bone mass, the World Health Organization (WHO) report defined diagnostic categories based on BMD measurements as follows:
a. Normal: t-score above −1.
b. Osteopenia: t-score between −1 and −2.5
c. Osteoporosis: t-score at or below −2.5

As used herein, the term ‘z-score’ refers to the number of standard deviations that a given subject's bone density differs from the average for the subject's age. The z-score may also be restricted to groups other than a particular age, for example by sex, race, or combinations of such.

There are also different z-scores depending on the group used as a reference (for example, the group could include everybody of the same age, or it could be limited to people with the same age, race, gender and weight).

Furthermore, an individual's t-score and z-score can vary for different bones, and hence can be reported for BMD measurements made at different places within the individual's body, e.g., one at the femoral neck, another at the total hip, and another at the spine.

As used herein, the term “annual change in patients”, interchangeably referred as “expected annual change” or “EAC,” to the change in the velocity of ultrasound waves through bones per year. The normal annual changes are approximately 3% or less loss in bone density for post-menopausal women, for

As used herein, the term “dedicated system” refers to any system adapted to perform a specific operation.

As used herein, the term “non-dedicated system” refers to any system which is not adapted to perform a specific operation (e.g., conventional computing means).

As used herein, the term “conventional computing means” refers, to a standard personal computer such as a commercially available desktop computer or a notebook computer that does not have any hardware (e.g. a special-purpose card) adapted for measurements related to bone sonometry or bone densitometry.

As used herein, the term “translating means” refers to any means adapted to transfer the results of measurements or calculations from a dedicated system to a non-dedicated system. Such translating means can be either hardwired or wireless. As a specific example relevant to the invention disclosed herein, translating means can be adapted to transfer the results of measurements of arrival times of ultrasonic pulses from a dedicated detector or measuring device to conventional computing means, such that the analysis of the differences in the arrival times is performed on the non-dedicated conventional computing means.

As used herein, the term “Omnisense” is used to describe the device described herein for performing measurements of the speed of sound within tissue located within a patient's body, especially hard tissue such as bone.

As used herein, with respect to bones, the term “strength” refers to the Young's modulus.

As used herein, the term “about” refers to a value within ±25% of the nominal value.

As used herein, the term “BMD” refers to bone mineral density.

As used herein, the term “BUA” refers to broadband ultrasound attenuation.

As used herein, the term “DXA” refers to dual-energy x-ray absorptiometry, a method for measuring bone mineral density.

As used herein, the term “SOS” refers to the speed of sound in a given medium.

As used herein, the term “SPA” refers to single-photon absorptiometry, another method for measuring bone mineral density.

As used herein, the term “SQV” refers to System Quality Verification.

In the ultrasonic measuring apparatus of the current invention, one or more ultrasonic transducers direct signals through a bone of the subject being tested. A pulse generator is coupled to at least one of the transducers causing these transducer(s) to generate an ultrasonic sound wave which is directed through the bone structure. The speed with which this pulse reaches a receiving transducer is used to measure the speed of sound (SOS) in the bone, which correlates with the density of the bone. The use of ultrasound is advantageous because it is non-invasive and is well-suited, e.g., to repeated measurements or studies performed during the course of medical treatment for an unrelated condition, since no ionizing radiation is used. The improved apparatus and method herein disclosed has the additional advantage of being operable with an ordinary personal computer rather than with a dedicated computer system. Thus a healthcare practitioner need only carry a palm-size or smaller measurement head, which may be connected to a local computer located, e.g., in a pharmacy, doctor's office, or hospital. The device is therefore made highly portable and of greatly reduced expense.

The ultrasonic bone analysis apparatus can also measure the rate of change of attenuation of ultrasound with frequency in the range of 200 to 600 kHz (“broadband ultrasound attenuation” or “BUA”), in addition to the speed of passage of acoustic waves through the bone. The BUA is a relative quantity calculated using a baseline signal as a reference of the transmitted signal entering the bone. The dependence of ultrasound attenuation on the frequency (i.e. the dispersion relations of ultrasound in bone) can be used, according to protocols well-known in the art, as a method supplementary to SOS measurements for calculation of bone mineral density and strength.

According to a preferred embodiment of the present invention, a bone densitometer is provided that provides a measure of bone density. One or more sources of acoustic waves at ultrasonic frequencies (“transmitters”) is provided in a head or wand that can be placed on the outside of the body. Such transmitters can be, e.g., piezoelectric elements. In this same head one or more measurement transducers (“receivers”) is provided that detects and measures ultrasound signals. One or more of the measurement transducers are placed close enough to one or more of the sources such that the first received ultrasonic pulse will result from direct reflection off the closest bone (i.e. without any contribution from conduction of the ultrasonic pulse through bone). In addition, one or more measurement transducers is placed far enough from one or more of the sources such that the first ultrasonic pulses received by these measurement transducers will necessarily involve some element of conduction through bone. By simple algebraic manipulations of the distances between sources and receivers, and times involved, the conduction paths through soft tissue can effectively removed from the measurement, allowing for precise determination of bone conduction velocity.

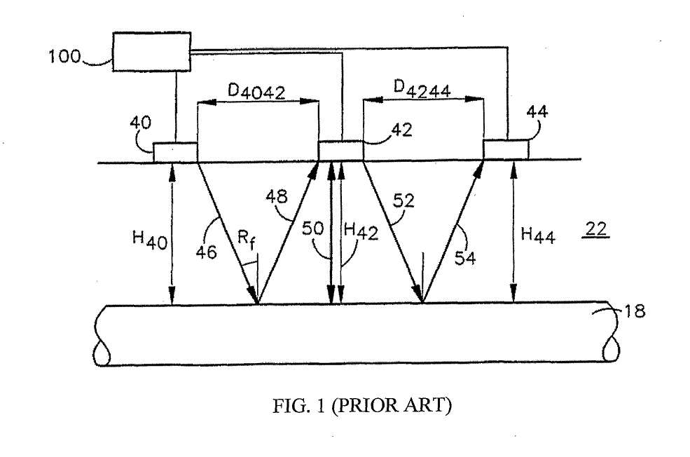

As detailed in WO97013145 ‘Ultrasonic device for determining bone characteristics’ (Kantorovich, Sunlight Medical Ltd) which is herein incorporated by reference, use of two or more sources is beneficial since the thickness of muscle or other soft tissue between the measurement device and the bone can be determined independently, and differences between detection times can be used to eliminate common conduction paths. Reference is now made to FIG. 1, which illustrates this method for removing the contributions to the signal by transmission through soft tissue. Transmitter 44 sends an ultrasonic pulse that is received by receivers at 40, 42. The first pulse received at 40 will take a path along 54, along the surface of the bone 18, and continue on path 46 to receiver 40. The first pulse received at 42 will be due to simple reflection off the bone, along paths 54, 52. Thus the difference between the times of arrival of the first signal at 40, 42 is due solely to the bone conduction path, without including the muscle or soft tissue conduction, as long as the paths 54, 46 are equal in length and composition. Further information can be gleaned by the time required for the signal to arrive at 42, since this includes muscle/soft tissue conduction only. Thus the path lengths 52, 54 can be determined. By using 40 as a transmitter and 42, 44 as receivers, remaining errors due to differences in soft tissue depth can be largely eliminated. A signal control unit 100 is provided to command and analyze the transmitter/detectors and the signals coming from them.

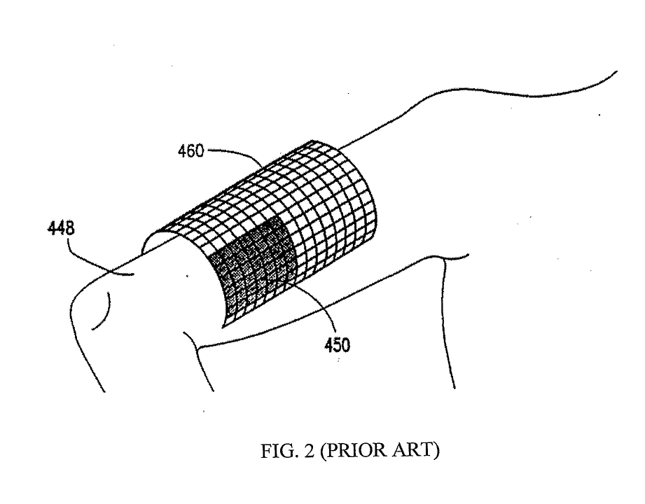

Reference is now made to FIG. 2, which shows a generalization of this principle to the use of a plurality of transmitter and receivers, as disclosed in U.S. Pat. No. 7,112,173 (Kantorovich, Sunlight Medical Ltd), which is incorporated herein by reference. Here, a matrix of transmitter/receiver elements 460 is placed around a body part such as upper arm 448. By means of timed pulses and subtraction as described above, a full map of bone density can be determined by such a device, at relatively high resolution of 2 mm or better in some cases. This resolution is high enough to detect hairline fractures and other inhomogeneities, especially since ultrasound conduction is greatly slowed by such. It should be recognized that this resolution is far higher than that available by other techniques such as single-source ultrasound, or X-ray methods.

Of particular significance is the fact that the SOS in a medium depends upon the Young's modulus of the medium involved. The longitudinal SOS (V_{L}) is given by the following equation:

$${V}_{L} = \sqrt{\frac{E ( 1 - σ )}{ρ ( 1 + σ ) ( 1 - 2 σ )}}$$

where E is the Young's modulus, σ is the Poisson ratio of lateral contraction to longitudinal extension, and ρ is the mass density of the material. Determination of the ratio of the Young's modulus to the density of the material is thus enabled by a measurement of longitudinal velocity. Since the Poisson ratio may be estimated independently, a further determination of either of the remaining variables (Young's modulus or density) suffices to determine the other.

It will be appreciated by one skilled in the art that the signal processing involved in this method is a key consideration in the implementation thereof. Control unit 100 has heretofore been located in a special signal processing card such as a PCI card, dropped into a standard desktop or portable PC.

A novel improvement of the current invention is the provision of a densitometer as described above with a more convenient location for signal processing, namely, within the measurement head itself. The processed signals can then be transferred to a standard PC for further analysis, obviating any need for a dedicated signal analysis card. This improvement is made possible by means of miniaturization of the basic processing elements and their incorporation into a single or small number of dedicated ASICs (application specific integrated circuits). These circuits deal with the realtime elements of the process, including signal production, sensing, and timing. By means of a standard communications protocol such as the USB protocol, information is conveyed to the PC for further processing.

It will be recognized that the above restructuring of the ultrasound diagnostic device allows it to be made highly portable and in fact independent of any particular PC. The device, consisting of measurement head and possibly associated hardware box, can be carried easily from location to location, such as from hospital to clinic. In each of these locations a PC is made available for the operator of the system. The user then simply connects the device to the USB port of the local PC (for example), runs the associated measurement software on the local PC, and begins the measurements desired. The executable file can be loaded onto the local PC from any portable storage medium such as a CD-ROM or disk-on-key (“thumb drive”) which is brought to the local PC, or by download from a predetermined web site (if the local PC has an internet connection). In this way, for the first time, bone sonometry/densitometry measurements are enabled without any necessity for transport of (or, for that matter, the purchase of) a dedicated PC, allowing for greatly improved portability of the device, as well as greatly reduced cost.

It is within the scope of the invention that communication between measurement head and computer be accomplished by any means known in the art, including but not limited to infrared, radiofrequency, USB, RS232, packet radio, FM, AM, CDMA, IRDA, wireless LAN means, 3G/4G, WLAN, WPAN, HSDPA, 3.9G, IEEE 802.11, IEEE 802.15, IEEE 802.16, HIPERLAN2, WiFi, WiMAX, HSPDA, BLUETOOTH, cable connection, GSM, GPRS, other cabled and wireless methods, and combinations thereof.

It is within the scope of the invention that due to its high precision the device of the current invention can be used for diagnosis of conditions involving relatively slow changes of bone density, bone mass, or bone strength. For example, after menopause women tend to undergo loss of bone mass. This loss of mass will occur over a period of years and therefore tends to be difficult to detect especially in its critical early stages. The early stages are particularly critical since it is extremely difficult to replace bone mass once lost, but it is possible to arrest this process if caught in time. Only by use of a sufficiently sensitive technique such as that disclosed herein is it possible to detect such small changes.

According to one embodiment of the invention herein disclosed, the noninvasive device is designed for the quantitative measurement of the signal velocity of ultrasonic waves propagating at multiple skeletal sites (e.g., the distal one-third of the radius, the proximal third phalanx, and the fifth metatarsal). The SOS provides an estimate of skeletal fragility. The output is also expressed as a t-score and a z-score, and can be used in conjunction with other clinical risk factors as an aid to the physician in the diagnosis of osteoporosis and other medical conditions leading to reduced bone strength and, ultimately, in the determination of fracture risk. Multiple skeletal site testing provides clinicians with alternatives if one site is not accessible and with additional skeletal information (i.e., from bones with different combinations of cortical and cancellous material and from weight bearing and non-weight bearing sites) that assists in diagnosing osteoporosis and risk fracture.

The SOS measurement obtained by the device has a low precision error (henceforth “LPE”). The measurement is sufficiently precise in comparison with the EAC that it is suitable for monitoring bone changes that occur in the early years following menopause (i.e., age range approximately 50-65 years).

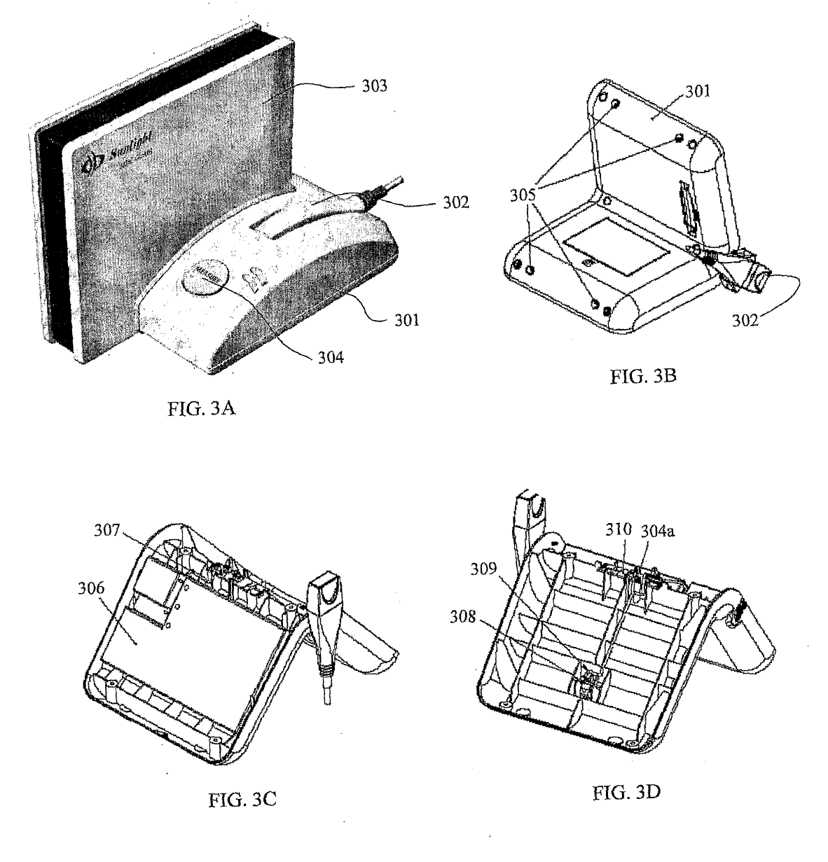

In some embodiments of the invention herein disclosed, a device is used that employs a hand-held probe designed to measure SOS values as the measurement head. Reference is now made to FIG. 3, which illustrates two embodiments of the hand-held probe and its associated base. FIG. 3A illustrates one embodiment of the device in which probe 302 rests within a slot located within probe base 301. Probe 302 comprises the transmitter(s) and receiver(s) as described above. Measurement button 304 activates the transmission of the ultrasonic waves as described above. Means for local storage of the results of measurements made by the probe, for transmitting the results of the measurements, and for interfacing the probe to an external computer are located within device body 303. FIGS. 3B-3D illustrate a second embodiment of probe base 301. In this embodiment, when not in use, probe 302 fits into a holder located on the side of base 301. FIG. 3B presents a bottom view of the base, illustrating the relative position of the holder and a plurality of feet 305 attached to the underside of the base. In preferred embodiments, the feet are made of a soft, non-slippery material such a rubber that allows the base to sit stably on a horizontal surface such as a table without risking scratching the finish of the surface on which the base sits. FIG. 3C presents a side view of base 301 with its cover removed. Control electronics 306 for the probe are located within the base. These control electronics are connected to the measure button via electrical connection 307. FIG. 3D presents the same view as FIG. 3C with the control electronics removed. In this view, processor 308, user interface card 310, and the data transfer connection 309 between them are visible. In addition, this view illustrates the relative location of measure button 304a in an internal view.

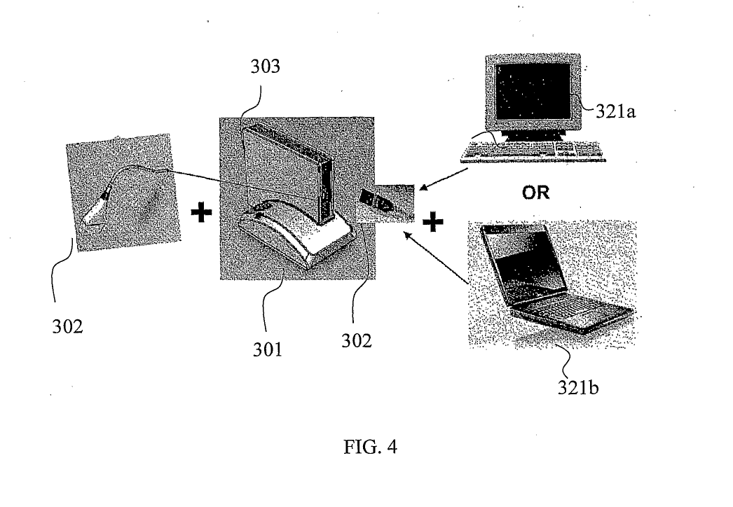

Reference is now made to FIG. 4, which shows how the assembly illustrated in FIG. 3 interfaces with a complete system. The probe and its associated electronics are connected to a main unit which can be, for example, a desktop (321a) or laptop (321b) computer. The connection may be of any type known in the art that is suitable for data transfer. In the embodiment illustrated, connection 320 is a standard USB interface connection.

In additional embodiments of the invention, the measurement head is provided in the form of a phased array of ultrasound sources and detectors. In some embodiments, this array is a cuff-shaped, possibly flexible probe containing multiple sources and sampling points for 3D reconstruction without any necessity for movement of the measurement head.

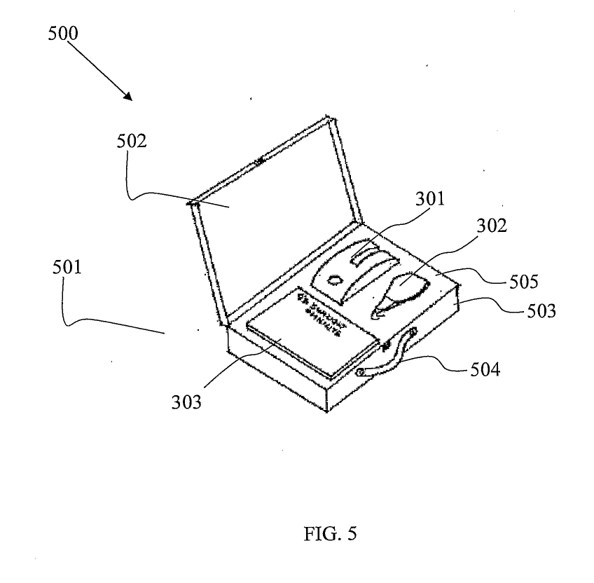

Because of the portability of the device, it can be provided to the user in the form of a kit. Reference is now made to FIG. 5, which shows one embodiment 500 of such a kit. The kit comprises closable case 501 comprising a lid 502, a bottom 503, and, in preferred embodiments, a handle 504. In preferred embodiments, the lid and bottom are joined together in a manner similar to cases (e.g. luggage) known in the art, for example, by a hinge. Bottom 503 is adapted to hold the components; in preferred embodiments, it contains a soft shock-absorbing material (e.g. polyurethane) into which slots or holes matching the components of the device are cut 505. In preferred embodiments of the device, the top is also filled with soft shock-absorbing material. In this manner, the device may be transported to the location at which it will be used with minimal risk of damage or loss during transport.

The probe is either connected to the Device Main Unit either via a hardwired or a wireless connection. During measurement, the probe is applied directly to the skin at locations such as the distal third of the radius. In preferred embodiments, a thin layer of gel is applied between the probe surface and the skin to facilitate good acoustic coupling. Any type of gel known in the art suitable for use with ultrasound probes in contact with a living creature may be used. Ultrasonic acoustic waves (in a preferred embodiment, having a center frequency of about 1.25 MHz) are produced by at least one transmitter located within the probe. In a preferred embodiment of the invention, the probe comprises two transmitters. The ultrasound waves are conducted along the bone and then detected by at least one receiver located in the same probe. As described above, the propagation time of the signals is used to calculate the SOS. The device's software compares the SOS result with the SOS of a young healthy population, as well as an age-matched population, using an embedded reference database (“normative database”), and reports the comparison in the form of a t-score and a z-score. In some embodiments of the invention, the normative database is updated with each new scan performed.

In a preferred embodiment of the present invention, a probe for performing a bone density measurement in non-dedicated conventional computing systems in a dedicated system especially useful for ultrasound bone sonometery, e.g., densitometry, is provided. The probe comprises (a) at least one ultrasound source for providing ultrasonic pulses; (b) a plurality of ultrasound detectors for measuring the differences in arrival times of the ultrasonic pulses; and, (c) translating means adapted to transfer the results of measurements of arrival times to conventional computing means, such that the analysis of the differences in the arrival times is performed by the non-dedicated conventional computing means. In other words, the actual tool that performs the analysis is the conventional PC computer and not a dedicated system.

In additional embodiments, the conventional computing means comprises: (1) a desktop or laptop personal computer-based Main Unit; (2) a video display; (3) a keyboard, further provided in some embodiments with an with integrated trackball or other integrated device for moving a cursor and/or pointer; (4) an SQV phantom; (5) in some embodiments, a foot pedal; (6) in some embodiments, a positioning gauge; (7) in some embodiments, a cushion hand rest; (8) in some embodiments, a set of earphones; and (9) a user guide.

The user interface comprises the keyboard and the optional integrated trackball, the video display monitor, the optional foot-pedal and an optional printer (which is not supplied with the device). The operator uses these accessories mainly to input patient information into the PC. These accessories are also used for entering other administrative input required in order to operate the system, such as the operator's I.D. and password, or the names of new operators or physicians. The software is adapted to display to the operator a list of previously measured patients, to enable the user to edit a patient information record, and to allow the operator to follow the progress of the measurement procedure.

An off-the-shelf printer may be used to generate a record of the patient information entered and the SOS Measurement Result as well as the corresponding t-scores and z-scores. The printer may also be used to print patient history data and SQV history data.

The System Quality Verification (SQV) procedure and a phantom, which is supplied with the system, are used to verify that the entire system is working properly. The phantom is composed of a homogenous hard polymeric material that transmits ultrasound signals at a known speed (typically approximately 2750 m s⁻¹ at room temperature). As a daily routine, the operator is requested to perform the SQV procedure. The SQV measurement procedure is performed in a manner similar to the measurement of the SOS of the radius and the same equations are used to compute the SOS value. The SQV procedure thus enables the user to verify that the measurement system is working properly and to calibrate measured SOS values against a known standard.

Two aids are supplied with the device: a radius measurement gauge and a hand rest. The gauge is made of a spring-loaded measuring band, connected at one end to a flat platform. The operator uses the gauge to measure the distance from the elbow to the tip of the third finger. Using a skin marker, a mark is drawn around the forearm at exactly the mid-point from the elbow to the third finger tip, which is the distal border of the region of interest.

Other accessories provided with the device include a set of earphones for listening to the on-line measurement methodologies.

The procedure for taking measurements with the device is performed according to the following steps: (1) opening a patient file; (2) marking the measurement position on the limb; (3) preparing the probe and the skin surface; (4) performing the actual bone measurements; and (5) reading the measurement results.

The measurement results are displayed on the monitor. The device reports the bone SOS, together with the t-score and z-score values, which are computed by the system's software using the patient's measured representative SOS value and the reference database. These values appear, together with a graphical display of the measurement results relative to the normative reference data, on the measurement results screen. A printout of the results can be obtained if a printer is connected to the Main Unit and the print button on the screen is activated. The physician may use these results in conjunction with other clinical risk factors as an aid in the diagnosis of osteoporosis and other medical conditions leading to reduced bone strength and, ultimately, in the determination of fracture risk. In order to monitor bone changes, the physician may recall the record of past measurements (measurement history) on the video display monitor or print them out.

Other operations can be performed with the help of the graphic user interface of the device. These include the daily SQV procedure, database management operations, defining system parameters, and other management operations.

The SOS measurements themselves are based on the basic principles and properties of the transmission of sound waves through a medium. Sound waves propagate in all directions from the transmitting transducer of the probe. Every molecule in the medium acts as a new transmitter, thus propagating the signal again in all directions in a manner similar to the Huygens principle for light. There are therefore many paths that the signal can follow from transmitter to receiver. The device detects the first signal to arrive at the receiving transducer. The time taken by the signal to travel between the transmitter and the receiver is the parameter measured by the device. This propagation time is a function of (1) the bone SOS; (2) the soft tissue SOS; (3) the average distance between the transducers and the bone; and (4) the angle of inclination between the surface of the bone and the line connecting the two transducers. The device software uses a proprietary algorithm to analyze these variables and to calculate the SOS through a particular region within a patient's body. The device's software then compares the SOS result with the SOS of a young healthy population, as well as an age-matched population, using an embedded reference database (informative database), and reports the comparison in the form of a t-score and a z-score.

In another embodiment of the invention herein disclosed, it is adapted for measuring the SOS in cartilage structures that are in the process of ossifying wherein the velocity is expected to increase as a function of the ossification during the human maturation process. Such acoustic signal velocity, for example, is measured in primary ossification centers such as the bones of the wrist or secondary ossification centers such as the distal regions of the ulna and radius.

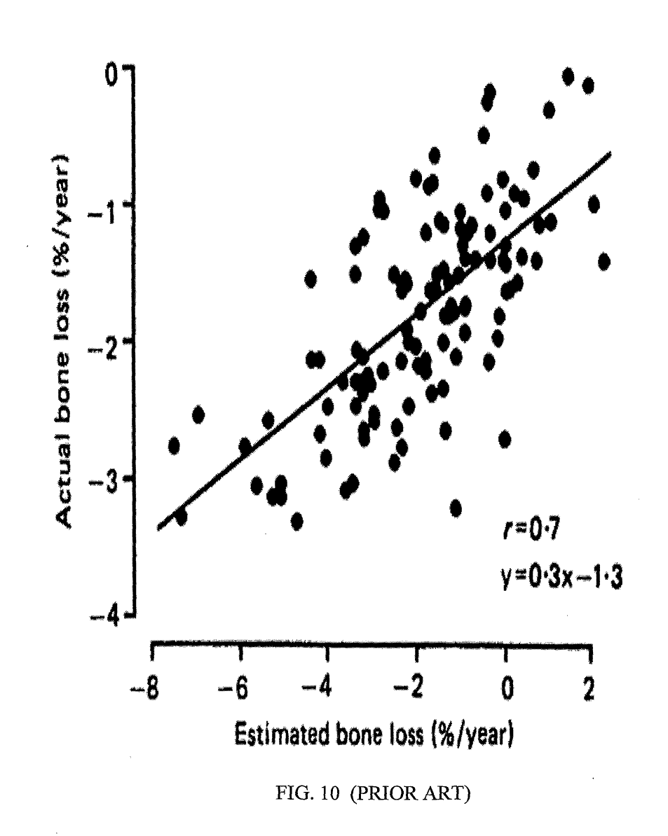

In another embodiment of the invention herein disclosed, the device is adapted for the measurement of bone age. Reference is now made to FIG. 10, which illustrates a method for measuring bone age known in the art, including: transmitting (120) acoustic energy into the body (150) of a subject; receiving (122) an acoustic signal from one or more structures including an ossification-actuated skeletal structure or a cranial structure that changes with age, responsive to the transmitted acoustic energy; analyzing (142) the acoustic signal to determine at least one effect of the structure on the signal; and estimating the age of the structure from the determined effect. It is within the scope of the current invention to use this method for bone age measurement, using the compact measurement head described above. The physical measurements are performed in the measurement head, while calculations, presentation, storage and the like are carried out with associated software on a PC. The same advantages listed above obtain also with the bone age measurement system; for example, once the measurement device is operable with a standard PC by means of standard communications devices (USB, wireless, etc.), the device operator can port the device without the computer. Thus the portability of the system is greatly improved.

In additional embodiments of the invention, the highly portable implementation provided in the current invention is included as part of a ‘diagnostic cart’ supplied with a number of such portable diagnostic devices. Such a device will be found to be highly useful for example in hospitals, clinics, pharmacies, and the like, since a number of important diagnostic tests can be performed quickly and easily from one highly portable unit. In addition to the ultrasound diagnostic device of the current invention such a diagnostic cart might include basic tools such as a sphygmomanometer, scale, height measurement ruler, blood analyzer, urine analyzer, optical chart, ear nose and throat inspection tools, stethoscope, rubber hammer, caliper, and the like. Such a cart can serve as a form of improved doctor's bag, including a full complement of simple tools as in a classic doctor's examination bag, but also provided with an array of modern electronic diagnostic tools, all preferably adapted for use with a single portable computer. Communication between the various electronic diagnostic devices and the computer can as above be accomplished by any number of standard communications protocols.

It is within the scope of the invention that a palm-held computer be provided with an ultrasound measurement head as described above, to allow a radical miniaturization of the entire system to the point that it may be carried in a user's pocket. Alternatively the measurement head may be supplied with a digital readout such as an LCD screen or the like to avoid the necessity even for a palm-held computer.

It is within the scope of the invention that the probe be made flexible.

As described in detail below, the device herein disclosed enables measurement of bone SOS with very high precision and accuracy. It is therefore within the scope of the invention to define a standard with regards to measurement accuracy. This higher accuracy method consists of measuring one or more of the group consisting of bone density, bone strength, and bone mass at a precision of at most 0.3% error in these parameters and at most 3 mm error in spatial resolution.

It is further within the scope of the invention to provide a standard concerning the frequency with which bone density changes be tested. One embodiment of this standard consists of yearly bone density tests, of a 0.3% precision and accuracy error or less in bone density, and at most a 3 mm error in spatial resolution. It is within the scope of the invention that part of this standard be to define groups to whom the standard applies. For instance, the National Osteoporosis Foundation as well as the American Association of Clinical Endocrinologists recommends all women aged 65 and over, as well as post-menopausal women who experience fractures, undergo a bone density test. They also suggest that younger women who have gone through menopause and have one or more risk factors (such as family history of spine fractures) undergo a bone density test as well.

It is therefore within the scope of the standard of the current invention that all post-menopausal women should get a bone density test within one year of menopause. This is useful because bone loss tends to speed up in the years after menopause, so getting a baseline idea of where one stands on entering menopause gives healthcare practitioners something to compare later scans to.

Particular risk groups required to undergo screening at least once a year according to the standard disclosed in the current invention include people who have suffered “fragility” fracture (defined as a bone break that occurs upon falling from a standing height of about 167 cm less); people who have been on high-dose corticosteroid medications to treat autoimmune diseases such as lupus; and women who have thyroid disease.

It is within the scope of the invention to provide a universal database updated with bone density and related information every time a scan is performed. This information is preferably catalogued on an online server, where it is useable for large-scale studies, longitudinal studies, and the like.

It is within the scope of the invention to provide a method of doing business as follows. The measurement device and associated software and hardware (if any) may be provided free of charge to an end user such as a pharmacy, clinic, doctor, or any other client. By use of a charge card, counter, passcode, or other means as will be obvious to one skilled in the art, a certain charge per screening or reading can be charged. Alternatively the machines may be leased, rented, or wholly owned by the pharmacy, clinic, doctor, patient, or other system user. In this way the initial cost of the system may be offset or eliminated altogether, making widespread adoption more possible. This is especially useful in situations such as kiosks or the like where casual passersby can get a free or low-charge bone density scan as is today common with free or low-cost blood pressure measurements. These devices may be placed in malls, public spaces, and the like.

It is within the scope of the invention to provide a method of doing business wherein the device is provided free of charge to a pharmaceutical or any private company whilst charging royalties for medications or referrals pursuant to use of the device.

According to one embodiment of the present invention, a method for leasing a bone densitometer is provided. The method comprises steps selected inter alia from: providing a bone densitometer as defined in any of the above; obtaining metering means for counting and storing the number of times the bone densitometer have been used; providing the densitometer to at least one user (with or without any additional fees for the densitometer); performing measurements of a patient's bone density; counting the number of times it has been used via the metering means; and, charging the user according to the count provided by the metering means. It is within the scope of the invention that the user can be any private user or private/public hospital, pharmacy or any combination thereof.

In other embodiments of the present invention, the method for leasing a bone densitometer comprises an additional step of prescribing medication to the patient if said bone density measurement is below a predetermined value, wherein royalties or commercial income as a function of the number and types of prescriptions are paid to the owner of the densitometer by the medication company.

In other embodiments of the present invention, the method for leasing a bone densitometer comprises an additional step of licensing bone sonometry, software to the at least one user; wherein the license is provided and can be used by at least one computer to which the license is provided.

According to another embodiment of the present invention, a method for creating a new database comprises information concerning the bone densities values of patients of different age, sex, race et cetera. The method comprises steps selected inter from: obtaining a densitometer according to any as described above; performing a bone density measurements to a patient; categorizing the patient according to parameters selected inter alia from age, sex, race or any combination thereof; and storing the bone density measurements either (i) in a local data base; or, (ii) uploading the bone density measurements to a communicable database.

In additional embodiments of the present invention, the method can additionally comprise steps of updating the database, receiving different measurements desired from the database, uploading new measurements into the database, performing different statistical operations on the database and hence obtaining variable information as for different classified patients.

In additional embodiments of the present invention, a bone densitometer is provided that can be detached from the computing platform that runs associated software. In this way the measurement device may be made highly portable, for example fitting into a small kit or even pocket sized. The user may attach this device to a local PC, for example a desktop or laptop PC, that is running software associated with the measurement device and adapted to control and receive measurements from it. By detaching the measurement head from the computing platform, a leap in portability is achieved.

In additional embodiments of the present invention, it is adapted for measuring of two or more acoustic signals of one or more ossification centers to determine bone age. The ratio of acoustic signals between two or more ossification centers is then used to determine bone age.

In additional embodiments of the present invention, it is adapted for measuring bone age, for example, the fibrocartilage of the pubic symphysis, skull suture ligaments and tooth and mandibular changes, by means of utilizing acoustic signals from structures associated with ossifying structures. In additional embodiments of the invention, these measurements are used to predict adult stature or other aspects of the maturation process. Such predictions are based on bone age derived by other methods known in the art. Alternatively or additionally, tracking of ossification in bone is used to detect and/or track the progress of various disease states and/or disorders, with, for instance, a more accurate profile than X-ray evaluation due to its non-ionizing nature, allowing frequent monitoring without harm.

In additional embodiments of the present invention, it is adapted for measuring bone age by means of parameters other than acoustic velocity, such as broadband ultrasound attenuation (BUA) and dispersion of the ultrasound signal, e.g., by correlating these parameters with the known BA assessment of a group of children. Signals reflected from bone are used to measure bone age, for example, are utilizable by measuring attenuation of backscatter intensity of the ultrasound signal.

In additional embodiments of the present invention, it is adapted for scanning or multi-beam measuring objects with unclear boundaries. Acoustic signals thus provide a spatial measure, for example, indicating a profile of velocity along a bone axis or a radial profile of an ossification center.

In additional embodiments of the present invention, it is adapted for analyzing, measuring or otherwise assessing different bones, different measures and/or different measurement systems to indicate different situations and/or for different bone ages or disease states.

In additional embodiments of the present invention, it is adapted for using an existing osteoporosis measurement device, possibly with minimal changes, to assess bone age. In one such embodiment, for example, a device designed for measuring osteoporosis in a finger is reprogrammed with a table that associates acoustic velocities with bone ages, rather than with states of osteoporosis.

In additional embodiments of the present invention, the transducers are modified specifically for application to growth centers. It is acknowledged in this respect that velocity limits used in osteoporosis measurement-devices are designed to obtain measurements from non-growth center areas.

It is also in the scope of the invention to disclose the aforementioned method for measuring bone age comprising: obtaining the aforementioned device as defined in any of the above or a system containing said device; transmitting acoustic energy into the body of a subject; receiving an acoustic signal from one or more structures including an ossification-actuated skeletal structure or a cranial structure that changes with age, responsive to the transmitted acoustic energy; analyzing the acoustic signal to determine at least one effect of the structure on the signal; and estimating the age of the structure from the determined effect.

It is also in the scope of the invention wherein the ossification-actuated skeletal structure comprises one or more areas undergoing ossification. Optionally, the ossification-actuated skeletal structure comprises one or more bones. Optionally, the ossification-actuated skeletal structure comprises one or more regions of cartilage. Optionally, the ossification-actuated skeletal structure comprises one or more regions of non-cartilage soft tissue. Optionally, the ossification-actuated skeletal structure comprises one or more regions of fibrocartilage.

It is also in the scope of the invention wherein the ossification-actuated skeletal structure comprises a region with one or more primary ossification centers. In preferred embodiments, the ossification-actuated skeletal structure comprises one or more of the bones of the wrist, the bones of the palm, the bones of the tarsus, the mandible.

It is also in the scope of the invention wherein the ossification-actuated skeletal structure comprises a region with one or more secondary ossification centers. Optionally, the ossification-actuated skeletal structure contains an epiphysis. Optionally the ossification-actuated skeletal structure comprises a region of one or more of a group consisting inter alia an ulna, a radius, a femur, a bone of a ray of an extremity.

It is also in the scope of the invention wherein the aforementioned step of receiving comprises step or steps of utilizing two or more different acoustic signals to provide a measure of bone age. As an option, the two or more acoustic signals are associated with the same bone. Optionally, the two or more acoustic signals are associated with paths in different bones. Optionally, the two or more acoustic signals are received from the same direction. Optionally, the two or more acoustic signals are received from the different directions.

Still referring to the aforementioned device and methods thereof, it is also in the scope of the invention wherein the signal passes through the one or more structures including an ossification-actuated skeletal structure.

Still referring to the aforementioned device and methods thereof, it is also in the scope of the invention wherein the signal echoes from the one or more structures including an ossification-actuated skeletal structure.

Still referring to the aforementioned device and methods thereof, it is also in the scope of the invention wherein the analysis of the signal is responsive to speed of sound from the one or more structures including an ossification-actuated skeletal structure.

Still referring to the aforementioned device and methods thereof, it is also in the scope of the invention wherein the analysis of the signal is responsive to broadband ultrasound attenuation from the one or more structures including an ossification-actuated skeletal structure.

Still referring to the aforementioned device and methods thereof, it is also in the scope of the invention wherein the analysis of the signal is responsive to dispersion of ultrasound from the one or more structures including an ossification-actuated skeletal structure.

Still referring to the aforementioned device and methods thereof, it is also in the scope of the invention wherein the analysis of the signal is performed, at least in part, in the frequency domain. Optionally, the analysis of the signal is performed, at least in part, in the time domain.

Still referring to the aforementioned device and methods thereof, it is also in the scope of the invention wherein the analysis of the signal is responsive to attenuation of an ultrasound signal in the one or more structures including an ossification-actuated skeletal structure. Optionally, the analysis is used to predict adult stature.

Still referring to the aforementioned device and methods thereof, it is also in the scope of the invention wherein the analysis is compared to a database having correlation with one or more members of a group consisting inter alia of conventional radiographs, CT images, MRI images and Nuclear Medicine scans, to provide an estimate of bone age

Still referring to the aforementioned device and methods thereof, it is also in the scope of the invention wherein the step of transmitting is provided by a scanning acoustic signal transmitter.

Still referring to the aforementioned device and methods thereof, it is also in the scope of the invention wherein the step of transmitting is provided by a multi-beam acoustic signal transmitter.

Still referring to the aforementioned device and methods thereof, it is also in the scope of the invention wherein the step of receiving provides two or more acoustic signal measures along an axis of the one or more structures including an ossification-actuated skeletal structure.

Still referring to the aforementioned device and methods thereof, it is also in the scope of the invention wherein the step of receiving provides two or more acoustic signal measures radially around the one or more structures including an ossification-actuated skeletal structure.

Still referring to the aforementioned device and methods thereof, it is also in the scope of the invention wherein the analysis is correlated with a known bone age measurement system.

Still referring to the aforementioned device and methods thereof, it is also in the scope of the invention wherein the analysis is responsive to a formula providing a correlation with a known bone age measurement system. Optionally, is responsive to at least one of speed of sound, broadband ultrasound attenuation, scattering and dispersion of acoustic signal through or from an ossification activated skeletal structure. Optionally, an estimate of bone age is responsive to time of flight of an acoustic signal between two transducers, with the ossification activated skeletal structure being situated intermediate to the transducers.

Still referring to the aforementioned device and methods thereof, it is also in the scope of the invention wherein the analysis separate formulas are used to correlate known bone age data with acoustic signals from males and females.

Still referring to the aforementioned device and methods thereof, it is also in the scope of the invention wherein the acoustic information is constructed into a database of bone age measurements. Optionally, the database is arranged according to one or more of sex, ethnic group, geographic location, nutrition and general inheritance.

Still referring to the aforementioned device and methods thereof, it is also in the scope of the invention wherein the database includes two or more measurements of one or more of the one or more structures including an ossification-actuated skeletal structure. Optionally, the database includes one or more measurements of two or more growth stages from the one or more structures including an ossification-actuated skeletal structure Optionally, the database includes one or more measurements of the one or more structures including an ossification-actuated skeletal structure in two or more populations.

Still referring to the aforementioned device and methods thereof, it is also in the scope of the invention wherein the received signals are compared to similar signals in a database to predict one or more of predict one or more of adult bone length, density, thickness and resilience and adult stature. Optionally, the received signals are compared to similar signals in a database to indicate a bone-growth related disorder. Optionally, the received signals are compared to similar signals in a database to track the progress of a bone-growth related disorder. Optionally, the received signals are compared to similar signals in a database to track hormone therapy in a growth stature disorder. Optionally, the received signals are compared to similar signals in a database to indicate one or more growth-plate related disease states, including osteogenic sarcoma, slipped growth plate, premature arrest of growth plate growth and inflammation of growth plate.

Still referring to the aforementioned device and methods thereof, it is also in the scope of the invention wherein the two or more acoustic measurements are made on a single subject and entered into the database. Optionally, the two or more acoustic measurements are compared to track one or more growth-related disorders, including precocious puberty, delayed puberty, rickets, kwashiorkor, hypoparathyroidism, pituitary dwarfism and diabetes.

Still referring to the aforementioned device and methods thereof, it is also in the scope of the invention wherein the two or more acoustic measurements are compared to track treatment of one or more growth-related disorders, including precocious puberty, delayed puberty, rickets, kwashiorkor, hypoparathyroidism, pituitary dwarfism and diabetes.

Still referring to the aforementioned device and methods thereof, it is also in the scope of the invention to disclose an apparatus and systems useful for estimating bone age. The sonometer-based apparatus and systems comprise inter alia the following modules: an acoustic transmitter and an acoustic receiver positioned on either side of one or more structures including an ossification-actuated skeletal structure; an electronic moveable gantry that adjusts the position of the acoustic transmitter and the acoustic receiver in relation to the ossification-actuated structure; a computer system that performs one or more functions of selected inter alia from a group consisting of positioning of the moveable gantry; controlling acoustic signals transmitted by the acoustic transmitter; receiving acoustic signals from the receiver responsive to the transmitted signals; and estimating the bone age responsive to the received signals.

Still referring to the aforementioned device and methods thereof, it is also in the scope of the invention wherein the device-based apparatus transmits and receives one or more acoustic signals linearly along an axis through the ossification-actuated structure.

Still referring to the aforementioned device and methods thereof, it is also in the scope of the invention wherein the device-based apparatus transmits and receives one or more acoustic signals radially around an axis through the ossification-actuated structure.

Still referring to the aforementioned device and methods thereof, it is also in the scope of the invention wherein the computer system controls the acoustic signal transmitter to provide an acoustic signal appropriate for the ossification-actuated structure.

Still referring to the aforementioned device and methods thereof, it is also in the scope of the invention wherein the computer system estimates the bone age responsive to one of more of broadband ultrasound attenuation, acoustic backscatter, dispersion of acoustic signal and speed of sound in the ossification-actuated structure.

Still referring to the aforementioned device and methods thereof, it is also in the scope of the invention wherein the computer system uses an imager to control the position of the acoustic signal receiver and the acoustic signal transmitter.

Still referring to the aforementioned device and methods thereof, it is also in the scope of the invention wherein the computer system contains a visual display to provide information on the bone age. Optionally, the visual display comprises a graph.

Still referring to the aforementioned device and methods thereof, it is also in the scope of the invention wherein the computer system is comprised in a computer network.

Still referring to the aforementioned device and methods thereof, it is also in the scope of the invention wherein the computer system comprises a neural network.

Still referring to the aforementioned device and methods thereof, it is also in the scope of the invention wherein the computer system compares the received acoustic signal to a database containing information of one or more acoustic signals from one or more structures including an ossification-actuated skeletal structure to provide an estimate of bone age.

Still referring to the aforementioned device and methods thereof; it is also in the scope of the invention wherein the computer system compares the received acoustic signal to a database containing information of one or more acoustic signals from one or more structures, including an ossification-actuated skeletal structure to predict stature.

Lastly, by referring to the aforementioned device and methods thereof, it is also in the scope of the invention wherein the computer system compares the received acoustic signal to a database containing information of one or more acoustic signals from one or more structures including an ossification-actuated skeletal structure to indicate, track or follow treatment of one or more of members of a group consisting inter alia a bone-growth related disorder, a growth plate disorder, and a growth related disorder.

### Example 1

In order to assess the properties of the device herein disclosed, a series of tests were performed to determine its precision, accuracy, compliance with international standards, and the effects of cleaning and disinfection on the device.

Two different in vivo precision tests for the device of the current invention were conducted. Included in these precision tests were: (1) a reproducibility study which involved the assessment of in vivo precision between different instruments, connecting slot configurations and probes; and (2) a reproducibility study which measured the in vivo precision between different operators and probes. The objectives of both studies were to estimate the variability, between device components and between operators, of SOS measurements of the distal one-third of the radius. The in vivo precision (reproducibility), expressed by the coefficient of variation (CV), ranged from 0.60% to 0.73%.

In addition, accuracy tests were performed as part of the testing of the device in order to verify compliance with the device's specifications that allow for line voltage variations as well as a range of environmental operating conditions. Two phantoms were measured under different environmental and line voltage conditions while changing the ultrasound probes and probe slot positions. The measured accuracy of the device was found better than +0.2% at both extremes of the SOS measurement range. The CV of the SOS result, computed from five successive measurements, was less than 0.1% in all of the different tests performed using either of the phantoms over a range of environmental conditions and operating line voltages tested.

The device was further tested to verify compliance of the device with acoustic output limits and requirements in accordance with (1) International Standard IEC 61157 “Requirements for declaration of the acoustic output of medical diagnostic ultrasonic equipment” (1993); (2) FDA's 510(k) Guidance: “Measuring and Reporting Acoustic Output of Diagnostic Ultrasound Medical Devices” (1985) and (3) FDA'S 510(k) Guidance: “Information for Manufacturers Seeking Marketing Clearance of diagnostic Ultrasound Systems and Transducers” (Sep. 30, 1997). The acoustic output test was performed based on the definitions and methods recognized by the National Electrical Manufacturers Association (NEMA), “Acoustic Output Measurement Standard for Diagnostic Ultrasound equipment”, UD-2 revision 2, NEMA (1997). The measured acoustic output levels of the device are summarized in Table 1, and are well below the limits specified in standard (3) above.

TABLE 1

| | |
|---|---|
| I_{SPTA, 3}[mW/CM²] | 6.5 |
| I_{SPPA, 3}[mW/CM²] | 3.7 |
| MI | 0.24 |
| W₍₀₎[mW] | 1.1 |

The ultrasound probe is considered a non-critical, reusable medical device which is applied only to intact skin, and therefore only low-level disinfection is required. The device's operator's manual includes a recommendation that users conduct disinfection procedures of the probe using Sporicidin Disinfectant Towelettes™. These towelettes have FDA 510(k) clearance for disinfection of medical devices (K904579), are EPA registered for “Hospital Disinfection” with AOAC testing protocols (Reg. No. 8383-7), and comply with OSHA Bloodborne pathogen Standard (29 CFR 1910.1030). Results from testing to determine the effects of disinfection methods on the probe characteristics demonstrated that a wiping method using Sporicidin Disinfectant Towelettes™ does not affect the probe parameters and the SOS measurement results and is, therefore, an acceptable method for low-level disinfection procedure.

### Example 2

Two clinical studies were undertaken in order to create normative reference databases for SOS in a Caucasian female population. The first of these was a multisector study performed in North America. This study was conducted on a group of Caucasian females between the ages of 20 and 90 years old by five investigators at five geographically diverse investigational sites in North America (4 in the U.S. and 1 in Canada). Potential subjects were identified by placing advertisements in the newspaper, contacting potential subjects from drivers' license listings, recruiting at college and university campuses, and recruiting at nursing homes. Eligible women had a negative history of osteoporosis fracture or chronic conditions affecting bone metabolism, and were not taking medications that affect bone metabolism. Of the 573 subjects recruited, 545 subjects were found eligible according to the inclusion/exclusion criteria of the study; SOS measurements at the distal third of the radius were performed on and analyzed for 521 of these subjects.

The mean SOS was 4083+146 m/sec with a range of 3532 to 4490. About 90% of the SOS measurements were between 3800 and 4300 m/sec. Over half of the measurements (52%) were between 4000 and 4200 m/sec. The results are summarized in Table 2.

TABLE 2

| Age | Mean ± SD |
|---|---|
| 20-29 | 4103 ± 107 |
| 30-39 | 4150 ± 93 |
| 40-49 | 4161 ± 130 |
| 50-59 | 4095 ± 131 |
| 60-69 | 3971 ± 141 |
| 70-79 | 3949 ± 125 |
| 80-89 | 3921 ± 149 |
| All | 4083 ± 146 |

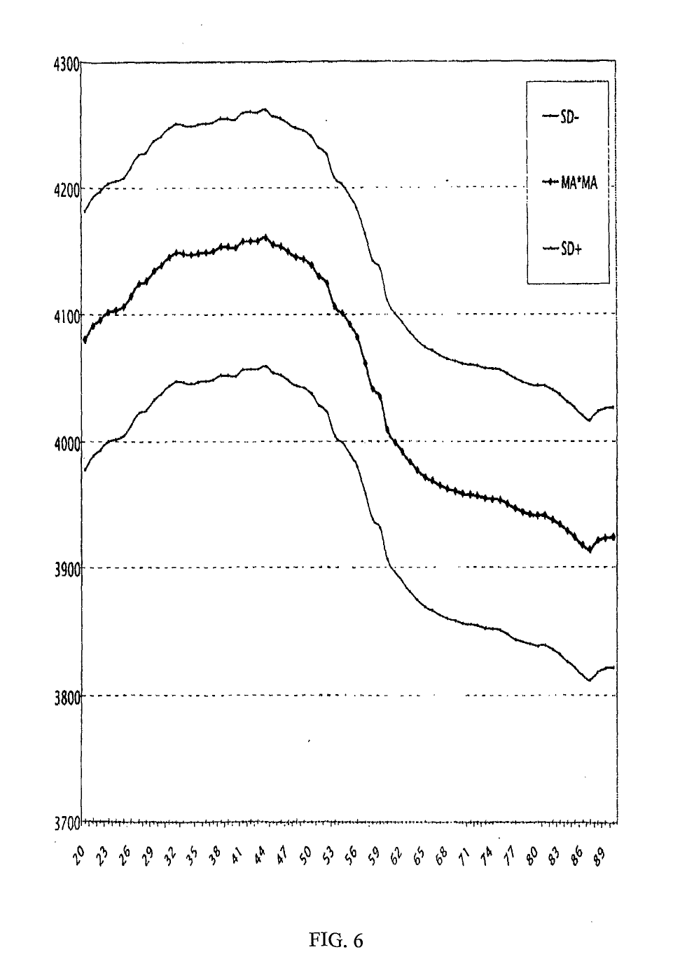

Reference is now made to FIG. 6, which depicts the moving average of the SOS results as a function of age. The moving average SOS increases to a peak of 4158 m s⁻¹ at the age of 41, with population standard deviation of 102 uses, and declines thereafter. The largest decline, about 1 5 m s⁻¹ y⁻¹, is observed around the age of 58, about eight years past the mean age of menopause. At older ages, 65 to 90, the decline slows down to about 2-5 m s⁻¹ y⁻¹. Linear regression models show that both a straight line and quadratic fit are highly significant (p<0.0001).

The moving average for the age of 41, and a representative standard deviation taken at the age decade around the peak SOS area, are used to calculate t-scores for each SOS measurement. The mean t-scores by age decade are shown in Table 3. The mean t-score of the entire eligible population in the study was −0.75±1.43 with a range of −6.16 to 3.24.

The mean t-score reached a low of −2.45 at age 80-89. This table also indicates the urgent of subjects in each age decade that had t-scores less than −2.5 (WHO criteria for osteoporosis) and those that had t-score between −2.5 and −1.0 (WHO criteria for osteonia). Among subjects aged 60-90 years, 35.0% had t-scores less than −2.5 and 42.3% had t-scores between −2.5 and −1.0.

TABLE 3

| | | | t < −2.5 | −2.5 < t < −1 |
|---|---|---|---|---|
| Age | N | mean ± SD | n (%) | n (%) |
| 20-29 | 92 | 0.56 ± 1.05 | 4 (4.3) | 24 (26.1) |
| 30-39 | 100 | 0.1 ± 0.92 | 1 (1.0) | 15 (15) |
| 40-49 | 102 | 0.01 ± 1.28 | 2 (2.0) | 15 (14.7) |
| 50-59 | 90 | 0.64 ± 1.28 | 7 (7.8) | 29 (32.2) |
| 60-69 | 64 | 1.84 ± 1.38 | 22 (34.4) | 24 (37.5) |
| 70-79 | 48 | 2.07 ± 1.23 | 16 (33.3) | 23 (47.9) |
| 80-90 | 25 | 2.34 ± 1.46 | 10 (40) | 11 (44) |
| All | 521 | 0.75 ± 1.43 | 62 (11.9) | 141 (27.1) |
| Range | | −6.16 to 3.24 | | |

No adverse events of any kind were reported in the course of this clinical study.

Conclusions: The North America normative database for Caucasian female population follows the classical curvature of bone densitometer, with minor variations, since bone properties other than mineral density are probed. The peak SOS value is observed at about the age of 41. A rapid decrease in SOS is further observed on or about the mean age of menopause, 51, reaching a maximal slope of about 15 m s⁻¹ y⁻¹ at the age range of 56 to 62. This change per year should be compared to the measurement precision of about 17 m s⁻¹. Being at about the same value, the Onmisense is shown to have a high sensitivity to change, thus making it suitable for measuring bone status in the first years acer menopause when bone changes are most pronounced. At older ages, the change per year moderates to a level of about 2-5 m s⁻¹ y⁻¹.

The prevalence of osteoporosis (in accordance with the World Health Organization definition) as measured by the SOS in the North American female population at the age of 60-69 was found to be about 35.5% which is comparable to the prevalence observed using axial DXA measurements.

### Example 3

The second study designed to create normative reference databases for SOS in a Caucasian female population was conducted in Caucasian females between the ages of 20 and 90 years old by a single investigator at Asaf-Harophe Medical Center, Israel. The eligibility criteria were met by 1,132 subjects who had their SOS measurements of the distal third of the radius taken. The mean age of the study subjects was 49.3±17.6 years with a range of 20 to 89 years. Each decade was roughly comparable in size except for the decade 40-49, in which there were 266 subjects. Sixty percent of the subjects in this study were pre-menopausal.

The mean SOS was 4082±151 m s⁻¹ with a range of 3510 to 4602. Ninety percent of the SOS measurements were between 3800 and 4300 m s⁻¹. Over half of the measurements (52.5%) were between 4000 and 4200 m/sec. Table 4 presents mean SOS result by age decade.

The moving average SOS increases to a peak of 4173 m s⁻¹ at the age of 39, with population standard deviation of 99 m s⁻¹, and declines thereafter. The largest decline, 15 m s⁻¹ y⁻¹, is observed around the age of 55, about four years past the mean age of menopause. At older ages, 65 to 90, the decline slows to about 5 m s⁻¹ y⁻¹. Linear regression models show that both a straight line and quadratic fit are highly significant (p<0.0001).

TABLE 4

| Age | Mean ± SD |
|---|---|
| 20-29 | 4108 ± 95 |
| 30-39 | 4161 ± 101 |
| 40-49 | 4167 ± 98 |
| 50-59 | 4115 ± 128 |
| 60-69 | 3989 ± 151 |
| 70-79 | 3931 ± 129 |
| 80-90 | 3879 ± 159 |
| All | 4082 ± 151 |

The mean t-scores by age decade are given in Table 5. The mean t-score for the study was −0.92±1.53 with a range of −6.70 to 4.33. The mean t-score reached a low of −2.97 at age 80-89.

This table also indicates the percent of subjects in each age decade that had t-scores of less than −2.5 (WHO criteria for osteoporosis) and those that had t-scores between −2.5 and −1.0 (WHO criteria for osteopenia). Among subjects aged 60-90, 44.9% had t-scores less than −2.5 and 34.5% had t-scores between −2.5 and −1.0.

TABLE 5

| | | | T < −2.50 | −2.50 < T < −1.0 |
|---|---|---|---|---|
| Age (years) | N | Mean ± SD | n (%) | n (%) |
| 20-29 | 182 | −0.65 ± 0.96 | 4 (2.2) | 60 (33.0) |
| 30-39 | 185 | −0.12 ± 1.02 | 3 (1.6) | 28 (15.1) |
| 40-49 | 266 | −0.06 ± 0.99 | 2 (0.8) | 37 (13.9) |
| 50-59 | 145 | −0.58 ± 1.30 | 12 (8.3) | 34 (23.4) |
| 60-69 | 160 | −1.86 ± 1.53 | 58 (36.2) | 56 (35.0) |
| 70-79 | 145 | −2.44 ± 1.31 | 68 (46.9) | 54 (37.2) |
| 80-90 | 49 | −2.97 ± 1.61 | 33 (67.3) | 12 (24.5) |
| All | 1132 | −0.92 ± 1.53 | 180 (15.9) | 281 (24.8) |
| Range | | −6.70 to 4.33 | | |

No adverse events of any kind were reported in the course of this clinical study.

Conclusions: The Israel normative database for Caucasian female population follows the classical curvature of bone densitometer similar to that of the North America normative database. Peak SOS value is observed at about the age of 39. A rapid decrease in SOS is further observed on or about the mean age of menopause, 51, reaching a maximal slope of about 15 m⁻¹ s⁻¹ y⁻¹ at the age range of 54 to 57, similar to the North America normative database previously described. This change per year may be compared to the measurement precision of about 17 m s⁻¹. Being at about the same value, the device of the current invention is again shown to have a high sensitivity to change, thus confirming the findings of the North American study that the device is suitable for measuring bone status in the first years after menopause when bone changes are most pronounced. At older ages, the change per year moderates to a level of about 5 m s⁻¹ y⁻¹.

The prevalence of osteoporosis (in accordance with the World Health Organization definition) as measured by the SOS in the Israeli female population at the age of 60-69 was found to be about 32% which is comparable to the prevalence observed using axial DXA measurements.

### Example 4

A cross-sectional case-control clinical study was performed at one investigational site in Israel in order to determine the ability of the device herein disclosed to discriminate osteoporosis hip fracture subjects from age matched non-fracture subjects and young healthy subjects, and to determine the fracture risk estimate.

Three different groups of subjects were recruited and analyzed in this study: 50 low trauma hip fracture (HF) subjects, 130 age-matched non-fracture subjects (NF) and 185 young healthy subjects (YF). The mean age for the hip fracture group was 76.1±6.0 years with a range of 65 to 85 years. The mean age for the non-fracture group was 71.5±5.2 with a range of 65 to 85 years. The mean age for the young healthy group was 40.6±3.0 with a range of 35 to 45 years.

The results of the SOS measurements performed in this study are given in Table 6. Hip fracture subjects had a mean SOS of 3861±149 m s⁻¹, while non-fracture subjects had a mean SOS of 3966±145 m s⁻¹. The difference between the two groups was statistically significant (p<0.0001). Young healthy subjects, on the other hand, had a mean SOS of 4165±96 m s⁻¹, which was greater than the mean SOS of both hip fracture subjects and elderly non-fracture subjects (p<0.0001 for both). The SOS distributions for the three study groups are also illustrated in FIG. 6. While there is a clear difference in the SOS distributions between the two elderly groups, there is an overlap as well in the range of 3800-3900 m s⁻¹, since it is likely that a significant proportion of the elderly subjects in the non-fracture group might also be osteoporotic.

TABLE 6

| | Hip Fracture | Elderly Non- | Young Healthy |
|---|---|---|---|
| Speed of Sound (m/sec) | n (%) | Fracture n (%) | n (%) |
| <3800 | 18 (36.0) | 15 (11.5) | 0 (0.0) |
| 3800-3899 | 12 (24.0) | 32 (24.6) | 0 (0.0) |
| 3900-3999 | 11 (22.0) | 35 (26.9) | 11 (5.9) |
| 4000-4099 | 5 (10.0) | 22 (16.9) | 33 (17.8) |
| 4100-4199 | 4 (8.0) | 19 (14.6) | 75 (40.5) |
| 4200-4299 | 0 (0.0) | 6 (4.6) | 52 (28.1) |
| 4300+ | 0 (0.0) | 1 (0.8) | 14 (7.6) |
| Total | 50 (100.0) | 130 (100.0) | 185 (100.0) |
| Mean ± SD | 3861 ± 149 | 3966 ± 145 | 4165 ± 96 |
| Range | 3490-4177 | 3582-4359 | 3901-4407 |
| T-test p-value | <0.0001 | — | <0.0001 |
| (vs. non-fracture) | | | |
| T-test p-value | <0.0001 | <0.0001 | — |
| (vs. young healthy) | | | |

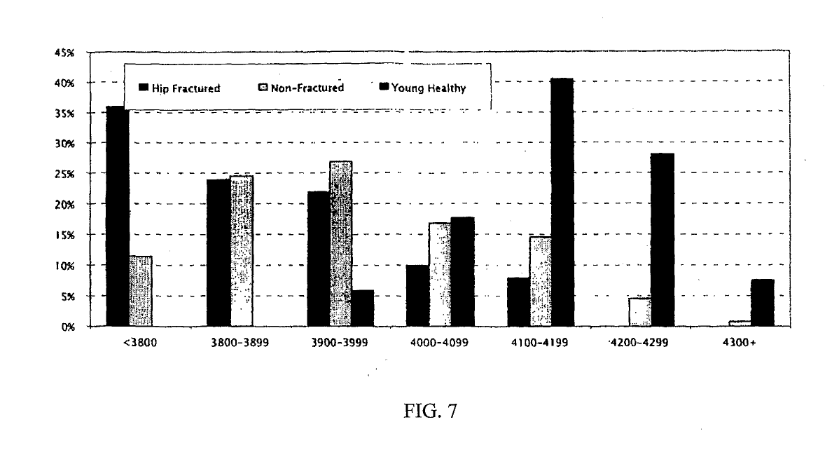

Reference is now made to FIG. 7, in which, the SOS distribution by Study Group is presented.

Table 7 shows the distribution of SOS t-scores for hip fracture and non-fracture subjects. Among hip fracture subjects, 70% (35/50) had t-scores <−2.5, while 39% (51/130) of non-fracture subjects and 1% (2/185) of young healthy subjects had t-scores <−2.5. Conversely, 10% (5/50) hip fracture subjects had t-scores >−1.0, while 24% (31/130) of non-fracture subjects and 85% (158/185) of young healthy subjects had t-scores >−1.0.

TABLE 7

| | | Elderly Non- | |
|---|---|---|---|
| | Hip Fracture | Fracture | Young Healthy |
| T-Score | n (%) | n (%) | n (%) |
| <−2.5 | 35 (70.0) | 51 (39.2) | 2 (1.1) |
| −2.5 to −1.0 | 10 (20.0) | 48 (36.9) | 25 (13.5) |
| >−1.0 | 5 (10.0) | 31 (23.8) | 158 (85.4) |
| Total | 50 (100.0) | 130 (100.0) | 185 (100.0) |
| Mean ± SD | −3.11 ± 1.52 | −2.06 ± 1.47 | −0.02 ± 0.98 |
| Range | −6.91 to 0.10 | −5.97 to 1.96 | −2.71 to 2.45 |

Table 8 presents a logistic regression analysis for hip fracture discrimination (i.e. comparing hip fracture subjects with elderly non-fracture subjects). This analysis indicates that the area under the ROC curve (AUC) is 0.63 (95% CI: 0.61-0.79) and the fracture odds ratio is 2.16 (95% CI: 1.46-3.19). The age- and BMI-adjusted AUC is 0.79 (95% CI: 0.73-0.84) and the age-adjusted odds ratio is 1.75 (95% CI: 1.15-2.65).

TABLE 8

| ROC | Odds ratio | p- | ROC | Odds ratio | |
|---|---|---|---|---|---|
| (95% CI) | (95% CI) | value | (95% CI) | (95% CI) | p-value |
| BMI & Age adjusted | | | BMI adjusted | | |
| 0.79 | 1.92 | 0.005 | 0.77 | 2.29 | 0.0002 |
| (0.73-0.84) | (1.22-3.02) | | (0.70-0.83) | (1.49-3.54) | |
| Age adjusted | | | Unadjusted | | |
| 0.75 | 1.75 | 0.009 | 0.69 | 2.16 | 0.0001 |
| (0.66-0.84) | (1.15-2.65) | | (0.61-0.79) | (1.46-3.19) | |

Table 9 shows the results of a logistic regression with fracture status as the dependent variable (excluding young healthy subjects) and SOS as the independent variable, adjusting for age and BMI. This analysis shows that for every 100 m s⁻¹ decrease in the SOS, the odds of fracture increase by about 50%, and that for every 162 m s⁻¹ decrease in the SOS, the odds of fracture double. Age and BMI are independent predictors of fracture risk: for every additional decade of age the risk of fracture increases by nearly 2.5 times, and for every BMI the risk of fracture increases by more than 25%.

TABLE 9

| | Parameter | Standard | | |
|---|---|---|---|---|
| Variable | Estimate | Error | Chi-Square | p-value |
| Intercept | −14.96 | 7.53 | 3.95 | 0.05 |
| Age | −0.09 | 0.04 | 6.72 | 0.01 |
| BMI | 0.23 | 0.06 | 14.27 | 0.0002 |
| SOS | 0.004 | 0.0015 | 7.94 | 0.005 |

No adverse events of any kind were reported in the course of this clinical study.

Conclusions: This case-control based study has shown that the device of the present invention can significantly discriminate between young and healthy subjects, who are at very low risk of any osteoporosis fracture, and a group of elderly subjects, who are known to be, on the average, at high risk of fracture. Moreover, the device was also found to significantly discriminate between osteoporosis hip fracture subjects and age-matched elderly non-fracture subjects.

This finding is noted despite a high likelihood that there are a significant number of osteoporosis subjects in the non-fracture group. The odds ratios found in this study, which can be considered fracture risk estimates, are comparable to those of other bone assessment devices.

This study results show that the SOS, as measured by the device disclosed herein, can significantly aid the physician diagnosing a patient for osteoporosis and determining the patient's risk of fracture.

### Example 5

A second clinical study was performed to determine whether the device herein disclosed can discriminate osteoporosis fracture subjects from age-matched non-fracture subjects and healthy young subjects, and estimate the risk of osteoporosis fracture. Four groups of subjects were enrolled and found eligible to be analyzed in the study: 94 hip fracture subjects (HF), 50 vertebral fracture subjects (VF), 41 wrist fracture subjects (WF), and 89 elderly non-fracture subjects (NF). All subjects were in the age range of 55 to 85. The study was conducted by one investigator at Rambam Medical Center, Israel.

The SOS measurement results are given in Table 10. Hip fracture subjects had a mean SOS of 3873±154 m s⁻¹, vertebral fracture subjects bad a mean SOS of 3877±144 m s⁻¹, wrist fracture subjects had a mean SOS of 3880±154 m s⁻¹, and non-fracture subjects had a mean SOS of 3953+±138 m s⁻¹. The mean SOS for all fracture subjects was 3878±154 m s⁻¹. All of the differences between the mean SOS of each of the fracture group and the mean SOS of the non-fracture group were statistically significant (p<0.01).

TABLE 10

| | Hip | Vertebral | Wrist | All | Elderly Non- |
|---|---|---|---|---|---|
| Speed of Sound | Fracture | Fracture | Fracture | Fracture | Fracture |
| (m/sec) | n (%) | n (%) | n (%) | n (%) | n (%) |
| <3800 | 32 (34.0) | 16 (32.0) | 14 (34.1) | 51 (32.1) | 15 (16.9) |
| 3800-3899 | 21 (22.3) | 8 (16.0) | 7 (17.1) | 32 (20.1) | 21 (23.6) |
| 3900-3999 | 20 (21.3) | 16 (32.0) | 10 (24.4) | 42 (26.4) | 22 (24.7) |
| 4000-4099 | 16 (17.0) | 7 (14.0) | 8 (19.5) | 24 (15.1) | 16 (18.0) |
| 4100-4199 | 4 (4.3) | 3 (6.0) | 1 (2.4) | 8 (5.0) | 12 (13.5) |
| 4200-4299 | 1 (1.1) | 0 (0.0) | 1 (2.4) | 2 (1.3) | 2 (2.2) |
| 4300+ | 0 (0.0) | 0 (0.0) | 0 (0.0) | 0 (0.0) | 1 (1.1) |
| Total | 94 (100.0) | 50 (100.0) | 41 (100.0) | 159 (100.0) | 89 (100.0) |
| Mean ± SD | 3873 ± 154 | 3877 ± 144 | 3880 ± 154 | 3878 ± 154 | 3953 ± 138 |
| Range | 3326-4246 | 3577-4149 | 3415-4206 | 3326-4246 | 3718-4325 |
| T-test p-value | <0.0001 | 0.003 | 0.01 | 0.0001 | — |
| (compared to | | | | | |
| non-fracture) | | | | | |

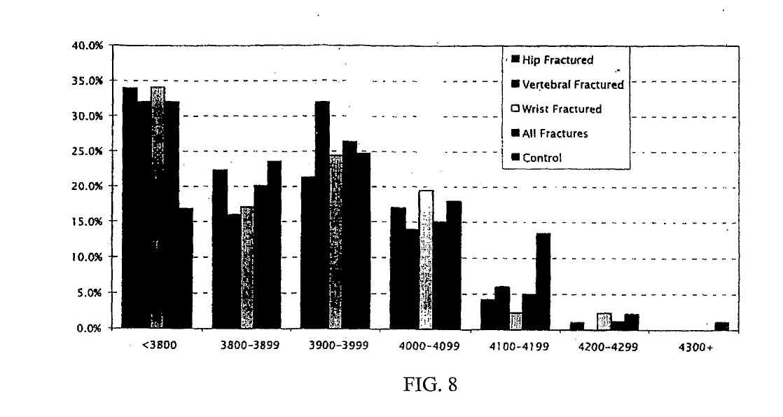

Reference is now made to FIG. 8, which illustrates the SOS distributions for the different study groups in the present study. While there is a clear difference in the SOS distributions between the fracture groups and the non-fracture group, there is considerable overlap in the range of 3800-3900 m s⁻¹.

Table 11 shows the distribution of SOS t-scores for each of the fracture groups and the non-fracture subjects. Among the different fracture groups, 60% of the hip fracture subjects, 52% of the vertebral fracture subjects and 54% of the wrist fracture subjects had t-scores <−2.5, as did 46% of non-fracture subjects. Conversely, less than 10% of each of the fracture groups had t-scores >−1.0, while 24% of non-fracture subjects had t-scores greater than −1.0.

TABLE 11

| | | Vertebral | Wrist | All | Elderly Non- |
|---|---|---|---|---|---|
| | Hip Fracture | Fracture | Fracture | Fracture | Fracture |
| T-Score | n (%) | n (%) | n (%) | n (%) | n (%) |
| <−2.5 | 56 (59.6) | 26 (52.0) | 22 (53.6) | 87 (54.7) | 41 (46.0) |
| −2.5 to 1.0 | 31 (33.0) | 20 (40.0) | 16 (39.0) | 59 (37.1) | 27 (30.3) |
| >1 | 7 (7.4) | 4 (8.0) | 3 (7.4) | 13 (8.2) | 21 (23.6) |
| Total | 94 (100.0) | 50 (100.0) | 41 (100.0) | 159 (100.0) | 89 (100.0) |
| Mean ± SD | −3.03 ± 1.55 | −2.99 ± 1.45 | −2.96 ± 1.56 | −2.98 ± 1.55 | −2.22 ± 1.39 |
| Range | −8.56 to 0.74 | −6.02 to −0.24 | −7.66 to 0.33 | −8.56 to 0.74 | −4.60 to 1.54 |

The logistic regression analysis for fracture discrimination (i.e. comparing all fracture subjects with elderly non-fracture subjects) presented in Table 12 indicates that the AUC is 0.63 (95% CI: 0.56-0.70) and the fracture odds ratio is 1.72 (95% CI: 1.29-2.30). The age- and BMI-adjusted AUC is 0.70 (95% CI: 0.63-0.77) and the age-adjusted odds ratio is 1.41 (95% CI: 1.04-1.93).

TABLE 12

| | | Vertebral | Wrist | All | Elderly Non- |
|---|---|---|---|---|---|
| | Hip Fracture | Fracture | Fracture | Fracture | Fracture |
| T-Score | n (%) | n (%) | n (%) | n (%) | n (%) |
| <−2.5 | 56 (59.6) | 26 (52.0) | 22 (53.6) | 87 (54.7) | 41 (46.0) |
| −2.5 to 1.0 | 31 (33.0) | 20 (40.0) | 16 (39.0) | 59 (37.1) | 27 (30.3) |
| >1 | 7 (7.4) | 4 (8.0) | 3 (7.4) | 13 (8.2) | 21 (23.6) |
| Total | 94 (100.0) | 50 (100.0) | 41 (100.0) | 159 (100.0) | 89 (100.0) |
| Mean ± SD | −3.03 ± 1.55 | −2.99 ± 1.45 | −2.96 ± 1.56 | −2.98 ± 1.55 | −2.22 ± 1.39 |
| Range | −8.56 to 0.74 | −6.02 to −0.24 | −7.66 to 0.33 | −8.56 to 0.74 | −4.60 to 1.54 |

Table 13 shows the results of a logistic regression with fracture status as the dependent variable and SOS as the independent variable adjusting for age and BMI. This analysis shows that the odds of hip, vertebral, wrist or any fracture increase by 50% for a decrease in SOS of 241 m s⁻¹, 127 m s⁻¹, 142 m s⁻¹ and 174 m s⁻¹ respectively. Furthermore the odds of hip, vertebral, wrist or any fracture double when the SOS decreases by 412 m s⁻¹, 217 m s⁻¹, 242 m s⁻¹, and 297 m s⁻¹, respectively.

TABLE 13

| | Parameter | Standard | | |
|---|---|---|---|---|
| Variable | Estimate | Error | Chi-Square | p-value |
| Intercept | −3.11 | 4.72 | 0.43 | 0.51 |
| Age | −0.09 | 0.02 | 16.67 | −0.0001 |
| BMI | −0.02 | 0.04 | 0.19 | 0.66 |
| SOS | 0.0023 | 0.0011 | 4.91 | 0.03 |

No adverse events of any kind were reported in the course of this clinical study.

Conclusions: This case-control based study has shown that the device of the current invention can significantly discriminate between subjects having any of the most common osteoporosis fractures (i.e., hip, vertebral and wrist fractures) and age matched non-fracture controls, even though the control group, being also formed of elderly subjects, likely comprises a significant number of osteoporosis subjects. A significant discrimination was similarly observed between each of the fracture subjects grouped according to their type of osteoporosis fracture, and the control group. The odds ratios found in this study which can be considered fracture risk estimates, are comparable to those found in the previous study, and also to those of other bone assessment devices.

These study results confirm once again, while widening the spectrum of the type of fractures, that the SOS as measured by the device herein disclosed can be used by physicians as a diagnostic tool for diagnosing a patient for osteoporosis and determining the patient's risk of fracture.

### Example 6

The cross-sectional studies reported in the two previous examples were very similar in many respects. Both studies had similar patient populations and recruited hip fracture subjects and healthy non-fracture subjects in the same age groups. Since hip fracture is the most important osteoporosis fracture from a personal, public health and economic point of view, it is important to obtain as accurate as possible an estimate of the ability of the device herein disclosed to discriminate between hip fracture and non-fracture subjects. To this end, the hip fracture and healthy non-fracture groups in the studies reported in the two previous examples have been pooled in order to arrive at a more precise estimate of the capabilities of the device herein disclosed.

The combined hip fracture group consists of 144 subjects, 50 from the study reported in Example 4 and 94 from the study reported in Example 5. The combined non-fracture group consists of 219 subjects, 130 from the study reported in Example 4 and 89 from the study reported in Example 5. Table 14 shows the distribution of SOS measurements for the combined hip fracture group and the combined non-fracture group. Hip fracture subjects had a mean SOS of 3869±152 m/sec, while non-fracture subjects had a mean SOS of 3960±142 m/sec (p<0.0001). As seen in this table, there is considerable overlap between the two groups in the range of 3800-4000 msec, since elderly subjects in the non-fracture group might also be osteoporosis.

TABLE 14

| | | Elderly Non- | |
|---|---|---|---|
| Speed of | Hip Fracture | Fracture | |
| Sound (m/sec) | n (%) | n (%) | p-value |
| <3600 | 3 (2.8) | 2 (0.9) | |
| 3600-3699 | 10 (6.9) | 0 (0.0) | |
| 3700-3799 | 37 (25.7) | 28 (12.8) | |
| 3800-3899 | 33 (22.7) | 51 (23.3) | |
| 3900-3999 | 31 (21.5) | 59 (26.9) | |
| 4000-4099 | 21 (14.6) | 38 (17.3) | |
| 4100-4199 | 8 (5.6) | 30 (13.7) | |
| 4200-4299 | 1 (0.7) | 9 (4.1) | |
| 4300+ | 0 (0.0) | 2 (0.9) | |
| Total | 144 (100.0) | 219 (100.0) | |
| Mean ± SD | 3869 ± 152 | 3960 ± 142 | <0.0001 |
| Range | 3326-4246 | 3582-4359 | |

Table 15 shows the distribution of SOS t-scores for the combined group of hip fracture subjects, as well as the combined group of non-fracture subjects. Among hip fracture subjects, 63% had t-scores <−2.5 while 42% of non-fracture subjects had t-scores <−2.5. Conversely, 8% of hip fracture Subjects had t-scores >−1.0, while 24% of non-fracture subjects had t-scores >−1.0.

TABLE 15

| | | Elderly Non- |
|---|---|---|
| | Hip Fracture | Fracture |
| T-Score | n (%) | n (%) |
| <−2.5 | 91 (63.2) | 92 (42.0) |
| −2.5 to −1.0 | 41 (28.5) | 75 (34.2) |
| >−1.0 | 12 (8.3) | 52 (23.7) |
| Total | 144 (100.0) | 219 (100.0) |
| Range | −8.56 to 0.10 | −5.97 to 1.96 |

The logistic regression analysis for hip fracture discrimination (i.e., comparing hip fracture subjects with elderly non-fracture subjects) presented in Table 16 indicates that the AUC is 0.67 (95% CI: 0.61-0.73) and the fracture odds ratio is 1.95 (95% CI: 1 .53-2.49). The age- and BMI-adjusted AUC is 0.76 (95% CI: 0.70-0.82) and the age-adjusted odds ratio is 1.54 (95% CI: 1.8-2.0%).

TABLE 16

| ROC | Odds ratio | p- | ROC | Odds ratio | |
|---|---|---|---|---|---|
| (95% CI) | (95% CI) | value | (95% CI) | (95% CI) | p-value |
| BMI & Age adjusted | | | BMI adjusted | | |
| 0.76 | 1.50 | 0.003 | 0.70 | 1.91 | 0.0001 |
| (0.70-0.82) | (1.15-1.96) | | (0.64-0.76) | (1.49-2.46) | |
| Age adjusted | | | Unadjusted | | |
| 0.75 | 1.54 | 0.001 | 0.67 | 1.95 | 0.0001 |
| (0.70-0.81) | (1.18-2.00) | | (0.61-0.73) | (1.53-2.49) | |

Table 17 shows the results of a multivariate logistic regression with fracture status as the dependent variable and SOS as the independent variable, adjusting for age and BMI. This analysis shows that for every 135 in s⁻¹ decrease in SOS the odds of fracture increase by about 50% and that for every decrease of 231 m s⁻¹ in SOS the odds of fracture doubles.

TABLE 17

| | Parameter | Standard | | |
|---|---|---|---|---|
| Variable | Estimate | Error | Chi-Square | p-value |
| Intercept | −4.79 | 4.27 | 1.26 | 0.26 |
| Age | −0.11 | 0.02 | 29.6 | <0.0001 |
| BMI | 0.12 | 0.04 | 10.3 | 0.001 |
| SOS | 0.0027 | 0.0009 | 8.68 | 0.003 |

Conclusions: The results from combining the two fracture studies show that the Omnisense can significantly discriminate between osteoporosis hip fracture subjects and age-matched non-fracture subjects even after controlling for age and BMI. The odds ratios found in this analysis are comparable to those of other bone assessment devices.

### Example 7

A further study was performed to determine the precision of the device, as measured by the coefficient of variation (CV). The SOS was measured at the distal third of the radius of each subject. The measurement was performed twice by each of three different operators. Probes were repositioned between each measurement. The CV was calculated using the SAS ANOVA procedure, which reports the overall mean, the mean square error (using subject-operator combination as a blocking factor) and the coefficient of variation (the mean square error divided by the mean). The CV was reported for all measurements as well as by operator and by menopausal status. The variance of each CV was also calculated so that 95% confidence intervals could be reported. Fifteen subjects were measured, 10 premenopausal women and 5 postmenopausal women.

A total of 45 pairs (15 subjects times 3 operators) of SOS measurements were used to compute the CVs. The overall CV was 0.40% (95% CI: 0.39% to 0.41%). For premenopausal women the CV was 0.29% and for postmenopausal women the CV was 0.57%.

A total of six different operators performed SOS measurements in this study. Their CVs ranged from 0.27% to 0.66%.

The coefficient of variation can also recalculated in two different “standardized CV” forms, SCV₁ and SCV₂. SCVI is computed by dividing the measured mean square error by 95% of the individual range, which is taken from the North America Normative Database determined as described above. The SCV₁ was found to be 1.8%. SCV₂ is computed by dividing the mean square error by the difference of the young healthy mean SOS (taken from the North America Normative Database) and that of the osteoporosis fracture mean SOS (the mean of the “All Fracture” group in the study reported in Example 5). SCV₂ is higher than SCV₁, and equals 5.9%.

No adverse events of any kind were reported in the course of this clinical study.

Conclusions: The in vivo precision of the device of the current invention, as measured by the coefficient of variation, is 0.40%. There were some relative differences in CV between premenopausal and postmenopausal subjects. Differences in precision between premenopausal subjects and postmenopausal subjects have been found in DXA measurements (postmenopausal CV higher than premenopausal CV) as well as in QUS measurements of the calcaneus (postmenopausal CV lower than premenopausal CV). There were also differences between CVs measured by different operators. Nevertheless, all CVs were well below 1% indicating good precision for all subgroups, and thus allowing for a meaningful assessment of patient status relative to the reference range.

The mean square error, about 17 m s⁻¹, is similar in magnitude to the average change per year which is observed during the first years of sharp decline in SOS post menopause, as described above. Thus the device of the current invention can provide precise estimates of bone status during this important time when bone changes are most pronounced.

1-64. (canceled)

65. A self-contained ultrasound bone sonometer device for performing multi-site bone density measurements of a region of a subject's body, said device comprising:
(a) a measurement head, said measurement head comprising:
i) an ultrasound source comprising an array of ultrasound transducers adapted for providing ultrasonic pulses;
ii) at least one measurement transducer adapted to measure the difference in arrival times of ultrasonic pulses; and,
iii) at least one dedicated data processing element adapted for determining differences in arrival times of ultrasonic pulses;
(b) means for transferring data from said at least one measurement transducer to said at least one dedicated data processing element; and,
(c) communication means adapted to transmit data from said dedicated data processing element to a non-dedicated computing means;
wherein a multi-site bone density measurement is obtained from said determination of differences in arrival times of ultrasonic pulses, and further wherein said determination is performed within said measurement head.

66. A probe for performing a bone density measurement, said probe comprising:
(a) at least one ultrasound source for providing ultrasonic pulses;
(b) a plurality of ultrasound detectors for measuring the differences in arrival times of said ultrasonic pulses;
(c) at least one dedicated data processing element adapted for determining differences in arrival times of ultrasonic pulses;
(d) means for transferring data from said at least one measurement transducer to said at least one dedicated data processing element; and,
(e) communication means adapted to transmit data from said dedicated data processing element to a non-dedicated computing means;
wherein analysis of said differences in said arrival times is performed by said non-dedicated computing means.

67. The probe according to claim 66, wherein at least one of the following is being held true (a) said probe comprises an array of said ultrasound sources and detectors adapted for being placed on or around a portion of a subject's body; (b) said probe is flexible; and any combination thereof.

68. An improved ultrasound bone sonometer device for multi-site bone density measurements, said device comprising:
(a) a cuff-like measurement head adapted to enclose at least part of the circumference of a region of a subject's body, said measurement head comprising an ultrasound source comprising an array of ultrasound transducers adapted for providing ultrasonic pulses;
(b) a dedicated data processing element; and,
(c) communication means adapted to transmit data from said dedicated processing element to a non-dedicated computing means;
wherein the improvement consists of the placement of (a) at least one measurement transducer adapted to measure the difference in arrival times of ultrasonic pulses and (b) at least one dedicated data processing element adapted for determining differences in arrival times of ultrasonic pulses within said measurement head.

69. The device according to claim 65, wherein at least one of the following is being held true (a) said dedicated data processing element is an ASIC; (b) said non-dedicated computing means is selected from the group consisting of server, PC, mobile communication means, workstation, personal digital assistant, laptop computer, desktop computer, tablet computing device, and any combination thereof; (c) said n-dedicated computing means comprise software adapted to perform at least one task chosen from the group consisting of (i) calculating the acoustic velocity of said ultrasonic pulses in said bone from the distances between said first, second, and third locations, and the difference between the time of propagation of an ultrasonic pulse from said first location to said second location and the time of propagation of an ultrasonic pulse from said first location to said third location; (ii) displaying the results of said acoustic velocity measurements; and (iii) storing the results of said acoustic velocity measurements; (d) said non-dedicated computing means are provided with processing software for analyzing said differences in said arrival times; and any combination thereof.

70. The device according to claim 65, wherein at least one of the following is being held true (a) said bone density measurement is presented in terms selected from the group consisting of BUA (Broadband Ultrasound Attenuation); SOS (Speed Of Sound); EPO (Expected Age of Osteoporosis); PAB (Physiological Age of Bone); TTO (Time To Osteoporosis); STI (Strength Index); RRF (Relative Risk of Fracture); pediatric capabilities; and (RFI) Risk Fracture Index; (b) said ultrasound detectors are adapted for measuring the differences in arrival times of said ultrasonic pulses; (c) said ultrasound source is adapted to provide (i) a first ultrasonic pulse transmitted along a transmission path extending from a first location to a second location, said transmission path comprising passage from said first location through soft tissue generally toward at least one bone, reflection from said at least one bone, and passage through said soft tissue generally away from said at least one bone and generally towards said second location; and, (ii) a second ultrasonic pulse transmitted along a transmission path from said first location to a third location, said transmission path comprising passage from said first location through soft tissue generally toward at least one bone, passage over the surface of said at least one bone, and passage through said soft tissue generally away from said at least one bone and generally towards a third location; and any combination thereof.

71. The device according to claim 65, wherein at least one of the following is being held true (a) said communication means are adapted to transmit data according to a communication protocol selected from the group consisting of: ZigBee, Bluetooth, IRDA, 3G, 4G, HSDPA, 3.9G, IEEE 802.11, IEEE 802.15.x, IEEE 802.16, HiperLAN, WiMAX, and GSM; (b) said communication means comprise a data connection selected from the group comprising USB, RS232, parallel cable, and wireless; (c) said communication means comprise networking means selected from the group comprising LAN, wireless LAN, PAN, and wireless PAN; (d) said communication means comprise communication methods selected from the group comprising CDMA, packet radio, and GPRS; (e) said communication means comprise means for transmitting data in at least one frequency range chosen from the group comprising visible light, infrared, microwave, and radiofrequency; and any combination thereof.

72. The device according to claim 68, further comprising a communicable database adapted for storage and retrieval of bone density readings.

73. The device according to claim 65, wherein said device is adapted to image or otherwise provide an analysis of at least one site within said measured region of a subject's body chosen from the group consisting of rigid sites and semi-rigid sites; further wherein said rigid site is a bone, bone-like biological tissue or region of a subject's body thereof; further wherein said semi-rigid site is a cartilage, cartilage-like biological tissue or region of a subject's body.

74. The device according to claim 65, wherein at least one of the following is being held true (a) said array of ultrasound sources and said ultrasound detector are located substantially on the same side of said region; (b) said array of ultrasound sources and said ultrasound detector are located on opposite sides of said region; (c) said array of ultrasound sources are constructed according to a phased arrays model; and any combination thereof.

75. The device according to claim 65, wherein said subject is selected from a group consisting of postmenopausal women, patients having “fragility” fracture, patients given corticosteroid medications, patients with thyroid disease, and patients of the age range of about 50 to about 65 years old.

76. A sonometer device for early detection of bone density changes in a subject in the age ranging from about 50 to about 65; said device comprising means for providing periodic bone density (PBD) readings with low precision error (LPE); wherein said precision error of said periodic bone density readings is of at least about an order of magnitude less than the expected annual change (EAC) in said subject, such that said early detection of said bone changes is provided.

77. The device according to claim 76, wherein at least one of the following is being held true (a) said precision error of said periodic bone density readings is less than 1% of the EAC in said subject, such that said early detection of said bone changes is provided; (b) said sonometer is a bone densitometer; and any combination thereof.

78. The device according to claim 76, wherein said means for providing PBD readings comprises:
(a) an array of ultrasound sources adapted for providing ultrasonic pulses;
(b) at least one measurement transducer adapted to measure the difference in arrival times of ultrasonic pulses; and,
(c) at least one dedicated data processing element adapted for determining differences in arrival times of ultrasonic pulses.

79. The sonometer according to claim 76, wherein said means for providing periodic bone density readings additionally comprises:
(a) at least one ultrasound source adapted for providing ultrasonic pulses;
(b) a plurality of ultrasound detectors adapted for measuring the differences in arrival times of said ultrasonic pulses; and,
(c) processing means for analyzing said differences in said arrival times.

80. The device according to claim 65, additionally comprising at least one selected from a group consisting of (a) means for limiting the amplitudes of said ultrasonic pulses to less than about 520 mV; (b) means for limiting the amplitudes of said ultrasonic pulses to about 400 mV; (c) means for normalizing the amplitudes of the signals received by said measurement head to about 400 mV; and any combination thereof.

81. A method for performing bone density measurements, comprising steps of:
(a) obtaining a portable ultrasound bone sonometer, comprising a measurement head provided with at least one ultrasound source, a plurality of ultrasound detectors, and at least one dedicated data processing element adapted for determining differences in arrival times of ultrasonic pulses;
(b) transmitting a plurality of ultrasonic pulses by said ultrasound source;
(c) detecting said a plurality of ultrasonic pulses by said detectors;
(d) measuring the differences in arrival times of said ultrasonic pulses;
(e) communicating said measurements of arrival times to at least one non-dedicated computing means; and,
(f) analyzing said differences in said arrival times;
wherein said step of analyzing is performed by said non-dedicated computing means.

82. A method for performing multi-site bone density measurements of a region of subject's body, said method comprising steps of:
(a) obtaining an ultrasound bone sonometer, said sonometer comprising (a) a cuff-like measurement head adapted to enclose at least part of the circumference of said region of said subject's body, said measurement head comprising: (i) an array of ultrasound sources adapted for providing ultrasonic pulses; (ii) at least one measurement transducer adapted to measure the difference in arrival times of ultrasonic pulses; and (iii) at least one dedicated data processing element adapted for determining differences in arrival times of ultrasonic pulses; (b) means for transferring data from said at least one measurement transducer to said at least one dedicated data processing element; and (c) means for transmitting data from said dedicated data processing element to a non-dedicated computing means;
(b) encircling said region of said subject's body by said flexible measurement head;
(c) transmitting ultrasonic pulses from said ultrasound sources to said multiple sites encircled within said region;
(d) detecting said ultrasonic pulses from said multiples sites encircled within said region; and,
(e) measuring the acoustic velocity of said ultrasonic pulses, thereby simultaneously performing said multi-site bone density measurements;
wherein said step of performing said multi-site bone density measurements is provided by said step of encircling said region of said subject's body by said flexible measurement head without having to relocate said measurement head from one site to another.

83. The method of claim 81, wherein said step of transmitting a plurality of ultrasonic pulses by said ultrasound source additionally comprises transmitting (i) a first ultrasonic pulse transmitted along a transmission path extending from a first location to a second location, said transmission path comprising passage from said first location through soft tissue generally toward at least one bone, reflection from said at least one bone, and passage through said soft tissue generally away from said at least one bone and generally towards said second location; and, (ii) a second ultrasonic pulse transmitted along a transmission path from said first location to a third location, said transmission path comprising passage from said first location through soft tissue generally toward at least one bone, passage over the surface of said at least one bone, and passage through said soft tissue generally away from said at least one bone and generally towards a third location; further wherein said step of measuring the acoustic velocity of said ultrasonic pulses additionally comprises a step of calculating the acoustic velocity of said ultrasonic pulses in said bone from the distances between said first, second, and third locations and the difference in time of propagation of an ultrasonic pulse from said first location to said second location and the time of propagation of an ultrasonic pulse from said first location to said third location.

84. The method of claim 81, additionally comprising at least one step selected from (a) displaying and storing said acoustic velocity measurements on said non-dedicated computing means; (b) selecting said computing means from the group consisting of: personal digital assistant, laptop computer, desktop computer, and tablet computing device; (c) presenting said bone density measurements in terms of a z-score; (d) presenting said bone density measurements in terms of a t-score; and any combination thereof.

85. The method according to claim 81, additionally comprising at least one step selected from (a) presenting said bone density measurements in terms selected from a group consisting of BUA (Broadband Ultrasound Attenuation); SOS (Speed Of Sound); EPO (Expected Age of Osteoporosis); PAB (Physiological Age of Bone); TTO (Time To Osteoporosis); STI (Strength Index); RRF (Relative Risk of Fracture); pediatric capabilities; and (RFI) Risk Fracture Index; (b) transmitting a plurality of ultrasonic pulses by said ultrasound source additionally comprises (i) transmitting a first ultrasonic pulse transmitted along a transmission path extending from a first location to a second location, said transmission path comprising passage from said first location through soft tissue generally toward at least one bone, reflection from said at least one bone, and passage through said soft tissue generally away from said at least one bone and generally towards said second location; and, (ii) transmitting a second ultrasonic pulse transmitted along a transmission path from said first location to a third location, said transmission path comprising passage from said first location through soft tissue generally toward at least one bone, passage over the surface of said at least one bone, and passage through said soft tissue generally away from said at least one bone and generally towards a third location; (c) analyzing additionally comprises a step of calculating the acoustic velocity of said ultrasonic pulses in said bone from the distances between said first, second, and third locations, and the difference between the time of propagation of an ultrasonic pulse from said first location to said second location and the time of propagation of an ultrasonic pulse from said first location to said third location; and any combination thereof.

86. The method of claim 81, wherein said step of analyzing additionally comprises steps of (a) displaying the results of said acoustic velocity measurements; and (b) storing the results of said acoustic velocity measurements.

87. The method according to claim 81, wherein at least one is being held true (a) said step of communicating additionally comprises a step of selecting a communication means adapted to transmit data according to a communication protocol selected from the group consisting of: ZigBee, Bluetooth, IRDA, 3G, 4G, HSDPA, 3.9G, IEEE 802.11, IEEE 802.15.x, IEEE 802.16, HiperLAN, WiMAX, and GSM; said step of communicating additionally comprises a step of selecting a communication means, said communication means comprising a data connection selected from the group comprising USB, RS232, parallel cable, and wireless; (c) said step of communicating additionally comprises a step of selecting a communication means, said communication means comprising networking means selected from the group comprising LAN, wireless LAN, PAN, and wireless PAN; (d) said step of communicating additionally comprises a step of selecting a communication means, said communication means comprising communication methods selected from the group comprising CDMA, packet radio, and GPRS; and any combination thereof.

88. The method according to claim 81, wherein said step of communicating additionally comprises steps of:
(a) selecting a communication means adapted to transmit data in at least one frequency range chosen from the group comprising visible light, infrared, microwave, and radiofrequency; and,
(b) transmitting data in said frequency range.

89. The method according claim 81, additionally comprising a step of calibrating said measurement; further wherein said step of calibrating said measurement comprises a step of measuring the propagation time of an ultrasonic pulse through a known length of a medium in which the speed of propagation of said ultrasonic pulse is known.

90. A method for early detection of bone density changes in subjects characterized in the age range of about 50 to about 65, comprising a step of performing periodic bone density readings at locations selected from the group consisting of the radius, the phalanx, the metatarsal and any combination thereof; wherein said readings are characterized by an LPE, and further wherein said precision error of said periodic bone density readings is at least about one order of magnitude less than the EAC, such that said early detection of said bone changes is provided; wherein said step of periodic bone density readings additionally comprises steps of: (a) transmitting a first ultrasonic pulse transmitted along a transmission path extending from a first location to a second location, said transmission path comprising passage from said first location through soft tissue generally toward at least one bone, reflection from said at least one bone, and passage through said soft tissue generally away from said at least one bone and generally towards said second location; (b) transmitting a second ultrasonic pulse transmitted along a transmission path from said first location to a third location, said transmission path comprising passage from said first location through soft tissue generally toward at least one bone, passage over the surface of said at least one bone, and passage through said soft tissue generally away from said at least one bone and generally towards a third location; and, (c) calculating the acoustic velocity of ultrasound in said bone from on the distances between said first, second, and third locations and the difference between the time of propagation of an ultrasonic pulse from said first location to said second location and the time of propagation of an ultrasonic pulse from said first location to said third location.

91. The method according to claim 90, additionally comprising steps of:
(a) limiting the amplitude of said ultrasonic pulses to less than 520 mV; and,
(b) normalizing the probes to approximately 400 mV.

92. The method according to claim 90, additionally comprising at least one step selected from (a) selecting said subjects from a group consisting of postmenopausal women, patients having “fragility” fracture, patients given corticosteroid medications, patients with thyroid disease, patients of the age range of about 50 to about 65 years old; (b) performing said periodic bone density readings at intervals selected from the group consisting of quarterly, biannually, and annually; and any combination thereof.

93. A method for measuring bone age, comprising steps of:
(a) providing a compact measurement head adapted to transmit an acoustic energy into the body of a subject;
(b) receiving an acoustic signal from one or more structures including an ossification-actuated skeletal structure or a cranial structure that changes with age, responsive to said transmitted acoustic energy;
(c) providing computing means adapted for analyzing the acoustic signal to determine at least one effect of said structure on said signal;
(d) providing communications means adapted to transmit acoustic signal information from said measurement head to said computing means; and,
(e) estimating the age of the structure from said determined effect with said computing means;
wherein bone age measurements are obtained in a system with separate measurement and computation means, allowing increased portability of said measurement head.

94. The device according to claim 68, wherein at least one of the following is being held true (a) said dedicated data processing element is an ASIC; (b) said non-dedicated computing means is selected from the group consisting of server, PC, mobile communication means, workstation, personal digital assistant, laptop computer, desktop computer, tablet computing device, and any combination thereof; (c) said n-dedicated computing means comprise software adapted to perform at least one task chosen from the group consisting of (i) calculating the acoustic velocity of said ultrasonic pulses in said bone from the distances between said first, second, and third locations, and the difference between the time of propagation of an ultrasonic pulse from said first location to said second location and the time of propagation of an ultrasonic pulse from said first location to said third location; (ii) displaying the results of said acoustic velocity measurements; and (iii) storing the results of said acoustic velocity measurements; (d) said non-dedicated computing means are provided with processing software for analyzing said differences in said arrival times; and any combination thereof.

95. The device according to claim 68, wherein at least one of the following is being held true (a) said bone density measurement is presented in terms selected from the group consisting of BUA (Broadband Ultrasound Attenuation); SOS (Speed Of Sound); EPO (Expected Age of Osteoporosis); PAB (Physiological Age of Bone); TTO (Time To Osteoporosis); STI (Strength Index); RRF (Relative Risk of Fracture); pediatric capabilities; and (RFI) Risk Fracture Index; (b) said ultrasound detectors are adapted for measuring the differences in arrival times of said ultrasonic pulses; (c) said ultrasound source is adapted to provide (i) a first ultrasonic pulse transmitted along a transmission path extending from a first location to a second location, said transmission path comprising passage from said first location through soft tissue generally toward at least one bone, reflection from said at least one bone, and passage through said soft tissue generally away from said at least one bone and generally towards said second location; and, (ii) a second ultrasonic pulse transmitted along a transmission path from said first location to a third location, said transmission path comprising passage from said first location through soft tissue generally toward at least one bone, passage over the surface of said at least one bone, and passage through said soft tissue generally away from said at least one bone and generally towards a third location; and any combination thereof.

96. The device according to claim 68, wherein at least one of the following is being held true (a) said communication means are adapted to transmit data according to a communication protocol selected from the group consisting of: ZigBee, Bluetooth, IRDA, 3G, 4G, HSDPA, 3.9G, IEEE 802.11, IEEE 802.15.x, IEEE 802.16, HiperLAN, WiMAX, and GSM; (b) said communication means comprise a data connection selected from the group comprising USB, RS232, parallel cable, and wireless; (c) said communication means comprise networking means selected from the group comprising LAN, wireless LAN, PAN, and wireless PAN; (d) said communication means comprise communication methods selected from the group comprising CDMA, packet radio, and GPRS; (e) said communication means comprise means for transmitting data in at least one frequency range chosen from the group comprising visible light, infrared, microwave, and radiofrequency; and any combination thereof.

97. The device according to claim 68, further comprising a communicable database adapted for storage and retrieval of bone density readings.

98. The device according to claim 68, wherein said device is adapted to image or otherwise provide an analysis of at least one site within said measured region of a subject's body chosen from the group consisting of rigid sites and semi-rigid sites; further wherein said rigid site is a bone, bone-like biological tissue or region of a subject's body thereof; further wherein said semi-rigid site is a cartilage, cartilage-like biological tissue or region of a subject's body.

99. The device according to claim 68, wherein at least one of the following is being held true (a) said array of ultrasound sources and said ultrasound detector are located substantially on the same side of said region; (b) said array of ultrasound sources and said ultrasound detector are located on opposite sides of said region; (c) said array of ultrasound sources are constructed according to a phased arrays model; and any combination thereof.

100. The device according to claim 68, wherein said subject is selected from a group consisting of postmenopausal women, patients having “fragility” fracture, patients given corticosteroid medications, patients with thyroid disease, and patients of the age range of about 50 to about 65 years old.

101. The device according to claim 68, additionally comprising at least one selected from a group consisting of (a) means for limiting the amplitudes of said ultrasonic pulses to less than about 520 mV; (b) means for limiting the amplitudes of said ultrasonic pulses to about 400 mV; (c) means for normalizing the amplitudes of the signals received by said measurement head to about 400 mV; and any combination thereof.

102. The device according to claim 76, additionally comprising at least one selected from a group consisting of (a) means for limiting the amplitudes of said ultrasonic pulses to less than about 520 mV; (b) means for limiting the amplitudes of said ultrasonic pulses to about 400 mV; (c) means for normalizing the amplitudes of the signals received by said measurement head to about 400 mV; and any combination thereof.

103. The method of claim 82, wherein said step of transmitting a plurality of ultrasonic pulses by said ultrasound source additionally comprises transmitting (i) a first ultrasonic pulse transmitted along a transmission path extending from a first location to a second location, said transmission path comprising passage from said first location through soft tissue generally toward at least one bone, reflection from said at least one bone, and passage through said soft tissue generally away from said at least one bone and generally towards said second location; and, (ii) a second ultrasonic pulse transmitted along a transmission path from said first location to a third location, said transmission path comprising passage from said first location through soft tissue generally toward at least one bone, passage over the surface of said at least one bone, and passage through said soft tissue generally away from said at least one bone and generally towards a third location; further wherein said step of measuring the acoustic velocity of said ultrasonic pulses additionally comprises a step of calculating the acoustic velocity of said ultrasonic pulses in said bone from the distances between said first, second, and third locations and the difference in time of propagation of an ultrasonic pulse from said first location to said second location and the time of propagation of an ultrasonic pulse from said first location to said third location.

104. The method of claim 82, additionally comprising at least one step selected from (a) displaying and storing said acoustic velocity measurements on said non-dedicated computing means; (b) selecting said computing means from the group consisting of: personal digital assistant, laptop computer, desktop computer, and tablet computing device; (c) presenting said bone density measurements in terms of a z-score; (d) presenting said bone density measurements in terms of a t-score; (e) calibrating said measurement; further wherein said step of calibrating said measurement comprises a step of measuring the propagation time of an ultrasonic pulse through a known length of a medium in which the speed of propagation of said ultrasonic pulse is known; and any combination thereof.

105. The method according to claim 82, wherein at least one is being held true (a) said step of communicating additionally comprises a step of selecting a communication means adapted to transmit data according to a communication protocol selected from the group consisting of: ZigBee, Bluetooth, IRDA, 3G, 4G, HSDPA, 3.9G, IEEE 802.11, IEEE 802.15.x, IEEE 802.16, HiperLAN, WiMAX, and GSM; said step of communicating additionally comprises a step of selecting a communication means, said communication means comprising a data connection selected from the group comprising USB, RS232, parallel cable, and wireless; (c) said step of communicating additionally comprises a step of selecting a communication means, said communication means comprising networking means selected from the group comprising LAN, wireless LAN, PAN, and wireless PAN; (d) said step of communicating additionally comprises a step of selecting a communication means, said communication means comprising communication methods selected from the group comprising CDMA, packet radio, and GPRS; and any combination thereof.

106. The method according to claim 82, wherein said step of communicating additionally comprises steps of:
(a) selecting a communication means adapted to transmit data in at least one frequency range chosen from the group comprising visible light, infrared, microwave, and radiofrequency; and,
(b) transmitting data in said frequency range.
